(19) United States
(12) Patent Application Publication
(10) Pub. No.: US 2022/0125899 A1
(43) Pub. Date: Apr. 28, 2022

(54) RNA CANCER VACCINES

(71) Applicant: ModernaTX, Inc., Cambridge, MA (US)

(72) Inventors: Ted Ashburn, Boston, MA (US); Kristen Hopson, Arlington, MA (US); Karen Keating, Brookline, MA (US); Joseph Senn, Cambridge, MA (US); Christine Swenson, Cambridge, MA (US); Benjamin Breton, Cambridge, MA (US); Shan Zhong, Cambridge, MA (US); Maija Garnaas, Somerville, MA (US)

(73) Assignee: ModernaTX, Inc., Cambridge, MA (US)

(21) Appl. No.: 17/291,947

(22) PCT Filed: Nov. 7, 2019
(86) PCT No.: PCT/US2019/060208

§ 371 Date: May 6, 2021

Related U.S. Application Data
(60) Provisional application No. 62/855,335, filed on May 31, 2019.
(60) Provisional application No. 62/813,900, filed on Mar. 5, 2019.
(60) Provisional application No. 62/757,057, filed on Nov. 7, 2018.

(51) Int. Cl. A61K 39/00; A61K 39/395; A61K 9/51; A61K 31/7105; A61K 48/00; A61P 35/00; C07K 16/28

(52) U.S. Cl. CPC A61K 39/0011; A61K 2039/876; A61K 9/5146; A61K 31/7105; A61K 48/005; A61P 35/00; C07K 16/2818; A61K 9/5123; A61K 2039/53; A61K 2039/54; A61K 2039/55555; A61K 2039/70; A61K 2039/82; A61K 2039/86; A61K 39/395

(57) ABSTRACT

The present application is related to a method of treating a cancer by administering to a human subject multiple doses of a mRNA cancer vaccine formulated as a lipid nanoparticle wherein the cancer vaccine comprises one or more mRNAs each having one or more open reading frames encoding 3-50 peptide epitopes, and wherein each of the peptide epitopes are portions of personalized cancer antigens or portions of cancer hotspot antigens. The present application further relates to a method of treating cancer by combining anti-cancer immunotherapy with the administration of the aforementioned mRNA cancer vaccine.

### RELATED APPLICATIONS

This application claims priority under 35 U.S.C. § 119(e) to U.S. provisional application No. 62/757,057, filed Nov. 7, 2018, U.S. provisional application No. 62/813,900, filed Mar. 5, 2019, and U.S. 62/855,335, filed May 31, 2019 the contents of each of which are incorporated herein by reference in their entireties.

### BACKGROUND OF INVENTION

Recent breakthroughs in cancer immunotherapy (e.g., checkpoint inhibitors and chimeric antigen receptor-T cell therapies) have demonstrated that powerful anti-tumor responses can be achieved by activating large numbers of T cells in a variety of cancer settings. Several checkpoint inhibitor biologic agents (e.g., anti-CTLA-4 [anti-cytotoxic T lymphocyte-associated antigen-4], anti-PD-1 [anti-programmed cell death protein 1], and anti-PD-L1 [anti-programmed death-ligand 1]) are currently approved for human use in several cancer types, including metastatic melanoma, non-small cell lung carcinoma and bladder carcinoma. These inhibitory receptors and their ligands play complementary roles in down-regulating adaptive immunity; PD-1/PD-L1 contributes to T cell exhaustion in peripheral tissues, and CTLA-4 inhibits earlier T cell activation events (Sharma and Allison 2015). Though it is clear that single agent checkpoint inhibitor therapy can provide significant benefit for some patients, many patients have incomplete or no response to therapy presenting a clear unmet need.

As tumors grow they acquire mutations, some of which create neoantigens that correlate with improved patient responses to immune checkpoint inhibitors. Several recent studies have shown the relevance of T-cell recognition of neoantigens in patients who respond to checkpoint blockade. Accumulating evidence suggests that checkpoint inhibitor efficacy is driven by blocking the negative signals generated by engagement of inhibitory receptors on T cells with their ligands on tumors and other immune cells, especially antigen presenting cells. The loss of inhibition following checkpoint blockade allows the patients' T cells to recognize neoantigens as foreign and this enhanced recognition leads to T-cell-mediated destruction of the tumor cells.

### SUMMARY OF INVENTION

Provided herein is a nucleic acid (e.g., ribonucleic acid (mRNA)) cancer vaccine having a maximized anti-cancer efficacy for a given length and comprising one or more nucleic acids that can direct the body's cellular machinery to produce nearly any cancer protein or fragment thereof of interest. Given that tumor mutations and their antigen presenting molecules (i.e., HLA) are unique to each patient, which necessitates a completely personalized vaccine approach to increase both the number and antitumor activity of a patient's T cells that recognize tumor-specific neoantigens, and the fact that no recurrent neoantigen peptide sequences have yet predicted responder patient populations, a completely personalized strategy is useful for capturing the full benefit of neoantigen vaccination. The methods of the disclosure provide vaccination with multiple, patient-specific neoantigens in order to improve clinical benefit for patients with a variety of cancer types. The methods disclosed herein also provide improved therapeutic efficacy of checkpoint inhibitors by the co-administration of vaccines which increase both the number and anti-tumor activity of a patient's T cells that recognize tumor-specific mutations/neoantigens.

In some aspects, the invention involves the use of a personalized mRNA cancer vaccine, having a unique mutational profile of an individual patient's tumor, matched to their specific HLA-type, in order to prime, activate, and expand a diverse population of neoantigen-specific T cells for the treatment of solid tumors.

The present disclosure, in some aspects, provides a method of treating a cancer in a human subject comprising administering to the human subject multiple doses of a mRNA cancer vaccine composition at a dosage of at least 0.04 mg, at least 0.13 mg, or at least 0.39 mg, or 0.04-0.13 mg, 0.13-0.39 mg, or 0.39-1.0 mg, 1.0-5.0 mg wherein the cancer vaccine composition comprises one or more mRNAs each having one or more open reading frames encoding 3-50, 20-40, 30-35 or 34 peptide epitopes, wherein each of the peptide epitopes are portions of personalized cancer antigens or portions of cancer hotspot antigens, formulated in a lipid nanoparticle formulation, thereby treating the cancer in the human subject. In some embodiments the cancer vaccine composition comprises one or more mRNAs each having one or more open reading frames encoding 34 peptide epitopes and wherein 29 epitopes are MHC class I epitopes and 5 epitopes are MHC class II or MHC class I and II epitopes.

In some embodiments, at least 2 doses of the cancer vaccine composition are administered to the subject. In some embodiments, at least 3 doses of the cancer vaccine composition are administered to the subject. In some embodiments, at least 4 doses of the cancer vaccine composition are administered to the subject. In some embodiments, at least 5 doses of the cancer vaccine composition are administered to the subject. In some embodiments, at least 6 doses of the cancer vaccine composition are administered to the subject. In some embodiments, at least 7 doses of the cancer vaccine composition are administered to the subject. In some embodiments, at least 8 doses of the cancer vaccine composition are administered to the subject. In some embodiments, at least 9 doses of the cancer vaccine composition are administered to the subject.

In some embodiments, the multiple doses of the cancer vaccine composition are administered 20-22 days apart. In some embodiments, the multiple doses of the cancer vaccine composition are administered 20 days apart. In some embodiments, the multiple doses of the cancer vaccine composition are administered 21 days apart. In some embodiments, the multiple doses of the cancer vaccine composition are administered 22 days apart.

In some embodiments, the cancer vaccine composition comprises one mRNA having one open reading frame encoding 3-50, 20-40, 30-35 or 34 peptide epitopes. In some embodiments, the cancer vaccine composition comprises one mRNA having one open reading frame encoding 10 peptide epitopes. In some embodiments, the cancer vaccine composition comprises one mRNA having one open reading frame encoding 12 peptide epitopes. In some embodiments, the cancer vaccine composition comprises one mRNA having one open reading frame encoding 14 peptide epitopes. In some embodiments, the cancer vaccine composition comprises one mRNA having one open reading frame encoding 15 peptide epitopes. In some embodiments, the cancer vaccine composition comprises one mRNA having one open reading frame encoding 16 peptide epitopes. In some embodiments, the cancer vaccine composition comprises one mRNA having one open reading frame encoding 18 peptide epitopes. In some embodiments, the cancer vaccine composition comprises one mRNA having one open reading frame encoding 20 peptide epitopes. In some embodiments the cancer vaccine composition comprises one or more mRNAs each having one or more open reading frames encoding 34 peptide epitopes and wherein 29 epitopes are MHC class I epitopes and 5 epitopes are MHC class II or MHC class I and II epitopes.

In some embodiments, the cancer vaccine composition comprises a first and second mRNA, wherein the first mRNA has one open reading frame encoding 3-20 peptide epitopes which are portions of personalized cancer antigens and wherein the second mRNA has one open reading frame encoding peptide epitopes that are portions of cancer hotspot antigens. In some embodiments, the cancer hotspot antigens comprise a KRAS G12 mutation or a KRAS G13 mutation or both mutations.

In some embodiments, the cancer vaccine composition is administered at a dosage of 0.04 mg to 2 mg. In some embodiments, the cancer vaccine composition is administered at a dosage of 0.04 mg to 1 mg. In some embodiments, the cancer vaccine composition is administered at a dosage of 0.39 mg to 2 mg. In some embodiments, the cancer vaccine composition is administered at a dosage of 0.39 mg to 1 mg.

In some embodiments, the minimum length of any peptide epitope is 8 amino acids. In some embodiments, the maximum length of any peptide epitope is 31 amino acids. In some embodiments, the minimum length of any or all of the peptide epitope is 13 amino acids. In some embodiments, the maximum length of any or all of the peptide epitope is 35 amino acids. In some embodiments, the length of any or all of the peptide epitopes is 25 amino acids.

In some embodiments, the one or more mRNA each comprise a 5′ UTR and/or a 3′ UTR.

In some embodiments, the one or more mRNA comprise at least one chemical modification. In some embodiments, the chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the one or more mRNA is fully modified.

In some embodiments, the peptide epitopes are in the form of a concatemeric cancer antigen comprised of 3-130 peptide epitopes. In some embodiments, the peptide epitopes are interspersed by cleavage sensitive sites. In some embodiments, each peptide epitope is linked directly to one another without a linker. In some embodiments, each peptide epitope is linked to one another with a single amino acid linker. In some embodiments, each peptide epitope is linked to one another with a short peptide linker. In some embodiments, each peptide epitope includes one or more SNP mutations; and/or a mutation causing a unique expressed peptide sequence. In some embodiments, the mRNA encoding the peptide epitopes is arranged such that the peptide epitopes are ordered to minimize pseudo-epitopes. In some embodiments, the ratio of class I MHC molecule peptide epitopes to class II MHC molecule peptide epitopes is at least 1:1, 2:1, 3:1, 4:1, or 5:1.

In some embodiments, the lipid nanoparticle formulation comprises a cationic lipid nanoparticle. In some embodiments, the lipid nanoparticle formulation comprises a molar ratio of about 20-60% ionizable amino lipid, about 5-25% non-cationic lipid, about 25-55% sterol, and about 0.5-15% PEG-modified lipid. In other embodiments the LNP is an ionizable amino lipid of Compound 1, wherein the non-cationic lipid is DSPC, the structural lipid is cholesterol, and the PEG lipid is PEG-DMG.

In some embodiments, the cancer vaccine composition prevents the human subject's cancer from recurring by provoking the immune system to develop a memory immune response against a cancerous tissue derived from an original lesion of the cancer.

In some embodiments, the vaccine composition is administered via intramuscular injection. In some embodiments, the vaccine composition is administered in 2 or more injections. In some embodiments, the vaccine composition is administered in a single injection.

In some embodiments, the human subject has a favorable response to the treatment method based on RECIST (Response Evaluation Criteria In Solid Tumors). In some embodiments, the human subject has a favorable response to the treatment method based on irRECIST (Immune-related Response Evaluation Criteria In Solid Tumors).

In some embodiments, the personalized cancer antigens are selected based on a next generation sequencing (NGS) analysis of the human subjects DNA from a tumor sample, relative to DNA from a blood sample.

The present disclosure, in another aspect, provides a method of treating a cancer in a human subject comprising administering to the human subject a combination therapy comprising (1) multiple doses of a mRNA cancer vaccine composition at a dosage of at least 0.04 mg, at least 0.13 mg, or at least 0.39 mg or 0.04-0.13 mg, 0.13-0.39 mg, or 0.39-1.0 mg, 1.0-5.0 mg, wherein the cancer vaccine composition comprises one or more mRNAs each having one or more open reading frames encoding 3-50, 20-40, 30-35 or 34 peptide epitopes, wherein each of the peptide epitopes are portions of personalized cancer antigens or portions of cancer hotspot antigens, formulated in a lipid nanoparticle formulation, and (2) multiple doses of an anti-cancer immunotherapy thereby treating the cancer in the human subject with the combination therapy.

In some embodiments the cancer vaccine composition comprises one or more mRNAs each having one or more open reading frames encoding 34 peptide epitopes and wherein 29 epitopes are MHC class I epitopes and 5 epitopes are MHC class II or MHC class I and II epitopes.

In some embodiments, the human subject is administered at least 0.04 mg of the vaccine composition. In some embodiments, the human subject is administered at least 0.13 mg of the vaccine composition. In some embodiments, the human subject is administered at least 0.39 mg of the vaccine composition. In some embodiments, the human subject is administered at least 1.0 mg of the vaccine composition. In some embodiments, the human subject is administered less than 2.0 mg of the vaccine composition.

In some embodiments, the lipid nanoparticle formulation comprises a cationic lipid nanoparticle. In some embodiments, the lipid nanoparticle formulation comprises a molar ratio of about 20-60% ionizable amino lipid, about 5-25% non-cationic lipid, about 25-55% sterol, and about 0.5-15% PEG-modified lipid. In other embodiments the LNP is an ionizable amino lipid of Compound 1, wherein the non-cationic lipid is DSPC, the structural lipid is cholesterol, and the PEG lipid is PEG-DMG.

In some embodiments, the anti-cancer immunotherapy is a checkpoint inhibitor. In some embodiments, the checkpoint inhibitor is pembrolizumab.

In some embodiments, the cancer vaccine composition is administered to the subject twice, thrice, four times, five times, six times, seven times, eight times, nine times, or 10 times. In some embodiments, the cancer vaccine composition is administered via intramuscular injection. In some embodiments, the anti-cancer immunotherapy is administered to the human subject at least 2 times. In some embodiments, the anti-cancer immunotherapy is administered to the human subject at least 3 times. In some embodiments, the anti-cancer immunotherapy is administered to the human subject at least 4 times. In some embodiments, the anti-cancer immunotherapy is administered to the human subject at least 5 times. In some embodiments, the anti-cancer immunotherapy is administered to the human subject at least 6 times. In some embodiments, the anti-cancer immunotherapy is administered to the human subject at least 7 times. In some embodiments, the anti-cancer immunotherapy is administered to the human subject at least 8 times. In some embodiments, the anti-cancer immunotherapy is administered to the human subject at least 9 times.

In some embodiments, the anti-cancer immunotherapy is administered to the human subject at least 2 times prior to the administration of the cancer vaccine composition. In some embodiments, the third dose of anti-cancer immunotherapy is administered to the human subject on the same day as a first dose of the cancer vaccine composition. In some embodiments, subsequent doses of the anti-cancer immunotherapy and the cancer vaccine composition are administered to the human subject on the same day.

In some embodiments, the cancer is selected from the group consisting of small cell lung cancer, urothelial cancer, colorectal cancer, endometrial cancer, stomach cancer, and gastro-esophageal junction cancer.

In some embodiments, the anti-cancer immunotherapy is administered every 20-24 days. In some embodiments, the anti-cancer immunotherapy is administered every 20 days. In some embodiments, the anti-cancer immunotherapy is administered every 21 days. In some embodiments, the anti-cancer immunotherapy is administered every 22 days. In some embodiments, the anti-cancer immunotherapy is administered every 23 days. In some embodiments, the anti-cancer immunotherapy is administered every 24 days.

In some embodiments, the anti-cancer immunotherapy is administered at a dose of 150-250 mgs. In some embodiments, the anti-cancer immunotherapy is 150 mgs. In some embodiments, the anti-cancer immunotherapy is 200 mgs. In some embodiments, the anti-cancer immunotherapy is 250 mgs.

In some embodiments the subject is selected for treatment based on a threshold microsatellite instability (MSI) value, tumor mutational burden (TMB) or T-cell inflamed gene expression profile (GEP). In some embodiments the GEP comprises PD-1, PD-L1 and PD-L2. In other embodiments the subject is selected for treatment based on high levels of PD-L1 and PD-L2. In other embodiments the subject is selected for treatment based high levels of PD-L1 and PD-1. In some embodiments the subject is selected for treatment based on a threshold GEP and TMB.

In some embodiments, the cancer vaccine is administered at a dosage level sufficient to deliver between 0.02-1.0 mg of the cancer vaccine to the subject. In some embodiments, the dose of the cancer vaccine is at least 0.04 mg. In some embodiments, the dose of the cancer vaccine is at least 0.13 mg. In some embodiments, the dose of the cancer vaccine is at least 0.39 mg. In some embodiments, the dose of the cancer vaccine is at least 1 mg. In embodiments the dose is less than 2.0 mg. In some embodiments, the cancer vaccine is administered to the subject twice, three times, four times, or more. In some embodiments, the cancer vaccine is administered by intradermal, intramuscular, intravascular, intratumoral, and/or subcutaneous administration. In some embodiments, the cancer vaccine is administered by intramuscular administration. In some embodiments, the cancer vaccine is administered with an anti-cancer agent, such as an antibody. In some embodiments, the cancer vaccine is administered with KEYTRUDA® (pembrolizumab).

In certain embodiments, the one or more nucleic acids encode 3-10 peptide epitopes, 5-10 peptide epitopes, 10-20 peptide epitopes, 20-30 peptide epitopes, 30-40 peptide epitopes, or 40-50 peptide epitopes peptide epitopes. In some embodiments, each of the peptide epitopes is encoded by a separate open reading frame. In some embodiments, the peptide epitopes are in the form of a concatemeric cancer antigen comprised of 3-130 peptide epitopes.

In some embodiments, one or more of the following conditions are met: a) the 3-130 peptide epitopes are interspersed by cleavage sensitive sites; and/or b) each peptide epitope is linked directly to one another without a linker; and/or c) each peptide epitope is linked to one or another with a single amino acid linker; and/or d) each peptide epitope is linked to one another with a short linker; and/or e) each peptide epitope comprises 8-31 amino acids and includes one or more SNP mutations; and/or f) each peptide epitope comprises 8-31 amino acids and includes a mutation causing a unique expressed peptide sequence; and/or g) the nucleic acid encoding the peptide epitopes is arranged such that the peptide epitopes are ordered to minimize pseudo-epitopes) no class II MHC molecule peptide epitopes are present.

In some embodiments, at least one of the peptide epitopes is a predicted T cell reactive epitope. In certain embodiments, at least one of the peptide epitopes is a predicted B cell reactive epitope. In some embodiments, the peptide epitopes comprise a combination of predicted T cell reactive epitopes and predicted B cell reactive epitopes. In certain embodiments, the peptide epitopes are predicted T cell reactive epitopes and/or predicted B cell reactive epitopes. In some embodiments, at least one of the peptide epitopes is a predicted neoepitope. In some embodiments, at least one nucleic acid has an open reading frame encoding at least a fragment of one or more traditional cancer antigens or one or more cancer/testis antigens.

In some embodiments, each nucleic acid is formulated in a lipid nanoparticle. In some embodiments, each nucleic acid is formulated in a different lipid nanoparticle. In certain embodiments, each nucleic acid is formulated in the same lipid nanoparticle.

In some embodiments, the total length of the one or more nucleic acids encodes a total protein length of 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, 900-1000 amino acids, 1000-1100 amino acids, or 1100-1200 amino acids. In some embodiments, the total length of the one or more nucleic acids is 1,235-2,924 nucleic acids. In some embodiments, the anti-cancer efficacy is calculated at least in part based on one or more factors selected from the group consisting of gene expression, RNA Seq, transcript abundance, DNA allele frequency, amino acid conservation, physiochemical similarity, oncogene, predicted binding affinity to a specific HLA allele, clonality, binding efficiency and presence in an indel. In certain embodiments, the one or more factors are inputted into a statistical model (e.g., a regression model (such as a linear regression model, a logistic regression model, a generalized linear model, etc.), a generalized linear model (such as a logistic regression model, a probit regression model, etc.), a random forest regression model, a neural network, a support vector machine, a Gaussian mixture model, a hierarchical Bayesian model, and/or any other suitable statistical model).

In some embodiments, the minimum length of any peptide epitope is 8 amino acids. In some embodiments, the maximum length of any peptide epitope is 31 amino acids. In some embodiments, the minimum length of any or all of the peptide epitope is 13 amino acids. In some embodiments, the maximum length of any or all of the peptide epitope is 35 amino acids. In some embodiments, the length of any or all of the peptide epitopes is 25 amino acids. In some embodiments, the total length of the vaccine encodes a total protein length of 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, 900-1000 amino acids, 1000-1100 amino acids, or 1100-1200 amino acids. In some embodiments, the total length of the one or more nucleic acids is 1,235-2,924 nucleic acids. In some embodiments, the score is calculated at least in part based on one or more factors selected from the group consisting of gene expression, RNA Seq, transcript abundance, DNA allele frequency, amino acid conservation, physiochemical similarity, oncogene, predicted binding affinity to a specific HLA allele, clonality, binding efficiency and presence in an indel. In certain embodiments, the one or more factors are inputted into a statistical model (e.g., a regression model (such as a linear regression model, a logistic regression model, a generalized linear model, etc.), a generalized linear model (such as a logistic regression model, a probit regression model, etc.), a random forest regression model, a neural network, a support vector machine, a Gaussian mixture model, a hierarchical Bayesian model, and/or any other suitable statistical model).

### BRIEF DESCRIPTION OF THE DRAWINGS

FIG. 1 is a graph showing the antigen-specific T cell responses for one human subject at the 0.13 mg dose level in a Phase I clinical trial.

FIG. 2 demonstrates the tumor mutation burden (TMB) of three human subjects at the 0.39 mg dose level in a Phase I clinical trial. The three human subjects are represented as larger points. The smaller points represent analyzed patient data.

FIG. 3 shows the gene expression of PD-1, PD-L1, and PD-L2 of three human subjects at the 0.39 mg dose level in a Phase I clinical trial. The larger points represent the three human subjects, while the smaller points show reported patient samples.

FIG. 4 is a graph showing the antigen-specific T cell response for one human subject with colorectal carcinoma (CRC) at the 0.39 mg dose level in a Phase I clinical trial.

FIG. 5 is a graph showing the antigen-specific T cell response for one human subject with melanoma at the 0.39 mg dose level in a Phase I clinical trial.

FIG. 6 is a graph showing the antigen-specific T cell response for one human subject with non-small cell lung carcinoma (NSCLC) at the 0.39 mg dose level in a Phase I clinical trial.

FIG. 7A shows data for adjuvant patients receiving mRNA monotherapy. FIG. 7B shows data for metastatic patients receiving the mRNA/pembrolizumab combination.

FIGS. 8A-8D show neoantigen specific CD8 T-cell responses in a patient post-dose 4 of mRNA. FIG. 8A: Increases in ex vivo (unexpanded) T-cell responses were detected against all neoantigen pulsed DC pools post vaccination. FIG. 8B: Increases in in vitro stimulated (IVS, expanded) T-cell responses were detected against all neoantigen pulsed DCs pools post vaccination. FIGS. 8C-8D: Greater than 3× (* in FIG. 8C) increases in neoantigen specific CD8 T-cells were detected post vaccination against 10 out of 18 class I targeted neoantigens included in a patient vaccine. All positive CD8 T-cell responses post vaccination were to neoantigens with high predicted binding affinity of <500 nm.

### DETAILED DESCRIPTION

In aspects, the invention relates to methods for improving efficacy of cancer therapy using personalized cancer vaccines. The vaccines increase both the number and antitumor activity of a subject's T cells, such that the subject can mount an effective T cell response that recognizes tumor-specific mutations and/or neoantigens. The tumor mutations and their antigen presenting molecules (i.e., HLA) are unique to each subject, and a personalized antigen/HLA strategy, such as the personalized cancer vaccines of the invention, maximize the personalized immune response. The design of the vaccine which incorporates multiple, subject specific neoantigens may improve clinical benefit for subjects with a variety of cancer types. In some aspects the personalized cancer vaccines may help to prevent the patient's cancer from recurring by instructing their immune system to better identify cancerous tissue derived from the original cancer lesion.

In other aspects the methods involve improving other anti-cancer therapies such as checkpoint inhibitor therapies. Checkpoint inhibitor efficacy may be driven by blocking the negative signals generated by engagement of these inhibitory receptors on T cells with their ligands on tumors and other immune cells, especially antigen presenting cells. The loss of inhibition following checkpoint blockade allows the subjects' T cells to recognize neoantigens as foreign. Combining the cancer vaccines of the invention with checkpoint inhibitor therapy, particularly in cancer patients with unresectable solid tumors, leads to T cell-mediated destruction of the tumor cells by increasing both the number and antitumor activity of a subject's T cells that recognize tumor-specific mutations/neoantigens. In a newly diagnosed subject, the checkpoint therapy, such as pembrolizumab, may begin as soon as possible. At the same time the subject's tumor sample can be screened for neoantigens and a personalized cancer vaccine may be designed and synthesized. As soon as the vaccine is ready, the subject may be started on the combination treatment. The checkpoint inhibitor may be administered together with the vaccine (i.e. on the same day) or they may be administered separately on different schedules.

The use of mRNA technology allows for induced production of a broad array of secreted, membrane-bound, and intracellular proteins in humans. Antigen-encoded mRNA is an attractive technology platform for neoantigen vaccination as an mRNA vaccine can deliver multiple neoantigens in a single molecule, a vaccine unique to each particular subject can be rapidly manufactured, and the neoantigens are endogenously translated and enter into the natural cellular antigen processing and presentation pathway. Moreover, this mRNA-based vaccine technology overcomes the challenges commonly associated with DNA-based vaccines, such as risk of genome integration or the high doses and devices needed for administration (eg, electroporation).

Each mRNA cancer vaccine consists of an mRNA encoding multiple neoantigens designed specifically to each individual subject's tumor mutanome and HLA type. This allows for the inclusion of the maximum number of neoantigens while both maintaining a sufficient amount of flanking sequence to facilitate both HLA Class I and Class II presentation of the peptides and retaining an mRNA construct length that can be reliably and rapidly manufactured.

Thus, embodiments of the present disclosure provide nucleic acid (RNA, such as mRNA) vaccines that include one or more nucleic acids having one or more open reading frames encoding peptide epitopes. As provided herein, nucleic acid cancer vaccines encoding peptide epitopes having different properties may be used to induce a balanced immune response, comprising cellular and/or humoral immunity. Methods of treating a patient having cancer with a cancer vaccine having a maximized anti-cancer efficacy for a given set of epitopes is also provided.

### Peptide Epitopes

The nucleic acid cancer vaccines of the disclosure may encode one or more peptide epitopes (which are portions of personalized cancer antigens). Portions of personalized cancer antigens are segments of personalized cancer antigens that are less than the full-length personalized cancer antigen. In one embodiment, the nucleic acid cancer vaccine is composed of open reading frames that may contain any number of peptide epitopes. In some embodiments the nucleic acid cancer vaccine is composed of open reading frames encoding 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 45 or more, 50 or more, 55 or more, 60 or more, 65 or more, 70 or more, 75 or more, 80 or more, 85 or more, 90 or more, 95 or more, 100 or more, 105 or more, 110 or more, 115 or more, 120 or more, 125 or more, 130 or more, 135 or more, 140 or more, 145 or more, 150 or more, 155 or more, 160 or more, 165 or more, 170 or more, 175 or more, 180 or more, 185 or more, 190 or more, 195 or more, or 200 or more peptide epitopes. In other embodiments the nucleic acid cancer vaccine is composed of open reading frames encoding 200 or less, 195 or less, 190 or less, 185 or less, 180 or less, 175 or less, 170 or less, 165 or less, 160 or less, 155 or less, 150 or less, 145 or less, 140 or less, 135 or less, 130 or less, 125 or less, 120 or less, 115 or less, 110 or less, 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, or 10 or less, or 5 or less peptide epitopes. In other embodiments the nucleic acid cancer vaccine is composed of open reading frames encoding up to 200, up to 195, up to 190, up to 185, up to 180, up to 175, up to 170, up to 165, up to 160, up to 155, up to 150, up to 145, up to 140, up to 135, up to 130, up to 125, up to 120, up to 115, up to 110, up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, up to 10 peptide epitopes, up to 5 peptide epitopes, or up to 3 peptide epitopes.

In some embodiments the nucleic acid cancer vaccines and vaccination methods described herein include open reading frames that encode epitopes or antigens based on specific mutations (neoepitopes) and/or those expressed by cancer-germline genes (antigens common to tumors found in multiple patients).

An epitope, also known as an antigenic determinant, as used herein is a portion of an antigen that is recognized by the immune system in the appropriate context, specifically by antibodies, B cells, or T cells. Epitopes may include B cell epitopes (e.g., predicted B cell reactive epitopes) and T cell epitopes (e.g., predicted T cell reactive epitopes). B-cell epitopes (e.g., predicted B cell reactive epitopes) are peptide sequences which are required for recognition by specific antibody producing B-cells. B cell epitopes (e.g., predicted B cell reactive epitopes) refer to a specific region of the antigen that is recognized by an antibody. T-cell epitopes (e.g., predicted T cell reactive epitopes) are peptide sequences which, in association with proteins on APC, are required for recognition by specific T-cells. T cell epitopes (e.g., predicted T cell reactive epitopes) are processed intracellularly and presented on the surface of APCs, where they are bound to MHC molecules including MHC class II and MHC class I molecules. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein. The sequences that interact with the antibody are situated next to each other sequentially on the protein, and the epitope can usually be mimicked by a single peptide. Conformational epitopes are epitopes that are defined by the conformational structure of the native protein. These epitopes may be continuous or discontinuous (i.e., may be components of the epitope can be situated on disparate parts of the protein, which are brought close to each other in the folded native protein structure).

Each peptide epitope may be any length that is reasonable for an epitope. In some embodiments, the length of each peptide epitope is not necessarily equal. In some embodiments, each peptide epitope in a nucleic acid cancer vaccine is a different length. In certain embodiments, at least two (e.g., at least 3, at least 4, at least 5, at least 6, at least 7, at least 8, at least 9, at least 10, at least 11, at least 12, at least 13, at least 14, at least 15, and up to and including all) of the peptide epitopes in a nucleic acid cancer vaccine are different lengths. In some embodiments, the length of at least one of the peptide epitopes is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 75, at least 80, at least 85, at least 90, at least 95, or at least 100 amino acids. In other embodiments, the length of at least one of the peptide epitopes is 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, 14 or less, 13 or less, 12 or less, 11 or less, 10 or less, 9 or less, 8 or less, 7 or less, 6 or less, 5 or less, 4 or less, 3 or less, or 2 or less amino acids. In other embodiments, the length of at least one of the peptide epitopes is up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, or up to 10 amino acids.

In some embodiments, each of the peptide epitopes encoded by the nucleic acid cancer vaccine may have a different length. In certain embodiments, at least one of the peptide epitopes has a different length than another peptide epitope encoded by the nucleic acid cancer vaccine. Each peptide epitope may be any length that is reasonable for an epitope.

In some embodiments, different percentages of peptide epitope lengths are encoded by the nucleic acids. All of the percentages described in the following listings may be approximate (i.e., within 5% of the stated amount). The use of the terms “approximate” and “about” is equivalent.

In some embodiments, the percentages of peptide epitope lengths encoded by the nucleic acids may be as follows: about 100%<15 amino acids, about 0%≥15 amino acids; about 95%<15 amino acids, about 5%≥15 amino acids; about 90%<15 amino acids, about 10%≥15 amino acids; about 85%<15 amino acids, about 15%≥15 amino acids; about 80%<15 amino acids, about 20%≥15 amino acids; about 75%<15 amino acids, about 25%≥15 amino acids; about 70%<15 amino acids, about 30%≥15 amino acids; about 65%<15 amino acids, about 35%≥15 amino acids; about 60%<15 amino acids, about 40%≥15 amino acids; about 55%<15 amino acids, about 45%≥15 amino acids; about 50%<15 amino acids, about 50%≥15 amino acids; about 45%<15 amino acids, about 55%≥15 amino acids; about 40%<15 amino acids, about 60%≥15 amino acids; about 35%<15 amino acids, about 65%≥15 amino acids; about 30%<15 amino acids, about 70%≥15 amino acids; about 25%<15 amino acids, about 75%≥15 amino acids; about 20%<15 amino acids, about 80%≥15 amino acids; about 15%<15 amino acids, about 85%≥15 amino acids; about 10%<15 amino acids, about 90%≥15 amino acids; about 5%<15 amino acids, about 95%≥15 amino acids; or about 0%<15 amino acids, about 100%≥15 amino acids.

In some embodiments, the peptide epitope lengths may be categorized in one of the following groups (for a total of 100%): 8-12 amino acids, 13-17 amino acids, 18-21 amino acids, 22-26 amino acids, or 27-31 amino acids. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 8-12 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 13-17 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 18-21 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 22-26 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the peptide epitopes encoded by the open reading frames of the nucleic acids may be 27-31 amino acids in length. Several non-limiting examples of the percentages of peptide epitope lengths encoded by the open reading frames of the nucleic acids follow.

In some embodiments, the peptide epitopes comprise at least one MHC class I epitope and at least one MHC class II epitope. In some embodiments, at least 10% of the peptide epitopes are MHC class I epitopes. In some embodiments, at least 20% of the peptide epitopes are MHC class I epitopes. In some embodiments, at least 30% of the peptide epitopes are MHC class I epitopes. In some embodiments, at least 40% of the peptide epitopes are MHC class I epitopes. In some embodiments, at least 0%, 60%, 70%, 80%, 90%, or 100% of the peptide epitopes are MHC class I epitopes. In some embodiments, none (0%) of the peptide epitopes are MHC class II epitopes. In some embodiments, at least 10% of the peptide epitopes are MHC class II epitopes. In some embodiments, at least 20% of the peptide epitopes are MHC class II epitopes. In some embodiments, at least 30% of the peptide epitopes are MHC class II epitopes. In some embodiments, at least 40% of the peptide epitopes are MHC class II epitopes. In some embodiments, at least 50%, 60%, 70%, 80%, 90% or 100% of the peptide epitopes are MHC class II epitopes. In some embodiments, the ratio of MHC class I epitopes to MHC class II epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class 1:MHC class II epitopes. In one embodiment, the ratio of MHC class I:MHC class II epitopes is 1:1. In one embodiment, the ratio of MHC class I:MHC class II epitopes is 2:1. In one embodiment, the ratio of MHC class I:MHC class II epitopes is 3:1. In one embodiment, the ratio of MHC class I:MHC class II epitopes is 4:1. In one embodiment, the ratio of MHC class I:MHC class II epitopes is 5:1. In some embodiments, the ratio of MHC class II epitopes to MHC class I epitopes is a ratio selected from about 10%:about 90%; about 20%:about 80%; about 30%:about 70%; about 40%:about 60%; about 50%:about 50%; about 60%:about 40%; about 70%:about 30%; about 80%:about 20%; about 90%:about 10% MHC class II:MHC class I epitopes. In one embodiment, the ratio of MHC class II:MHC class I epitopes is 1:1. In one embodiment, the ratio of MHC class II:MHC class I epitopes is 1:2. In one embodiment, the ratio of MHC class II:MHC class I epitopes is 1:3. In one embodiment, the ratio of MHC class II:MHC class I epitopes is 1:4. In one embodiment, the ratio of MHC class II:MHC class I epitopes is 1:5. In some embodiments, at least one of the peptide epitopes of the cancer vaccine is a B cell epitope. In some embodiments, one or more predicted T cell reactive epitope of the cancer vaccine comprises between 8-11 amino acids. In some embodiments, one or more predicted B cell reactive epitope of the cancer vaccine comprises between 13-17 amino acids.

The cancer vaccine of the disclosure, in some aspects comprises an mRNA vaccine encoding multiple peptide epitope antigens arranged with a single amino acid spacer between the peptide epitopes, a short linker between the peptide epitopes, or directly to one another without a spacer between the peptide epitopes. The multiple epitope antigens may include a mixture of MHC class I epitopes and MHC class II epitopes.

The nucleic acid cancer vaccine of the disclosure, in some aspects, comprises a nucleic acid encoding one or more peptide epitopes that include a mutation causing a unique expressed peptide sequence. In some embodiments, a mutation causing a unique expressed peptide sequence may be, but is not limited to, an insertion, deletion, frameshift mutation, and/or splicing variant. In some embodiments, the nucleic acid cancer vaccine encodes multiple peptide epitope antigens including one or more single nucleotide polymorphism (SNP) mutations with flanking amino acids on each side of the SNP mutation. In some embodiments, the number of flanking amino acids on each side of the SNP mutation may be 5, 6, 7, 8, 9, 10, 11, 12, 13, 14, 15, 16, 17, 18, 19, 20, 22, 24, 26, 28, or 30. In some embodiments, the SNP mutation is centrally located and the number of flanking amino acids on each side of the SNP mutation is approximately the same. In other embodiments, the SNP mutation does not have an equivalent number of flanking amino acids on each side. In an embodiment, an epitope of the cancer vaccine comprises an SNP flanked by two Class I sequences, each sequence comprising seven amino acids. In another embodiment, an epitope of the cancer vaccine comprises a SNP flanked by two Class II sequences, each sequence comprising 10 amino acids. In some embodiments, an epitope may comprise a centrally located SNP and flanks which are both Class I sequences, both Class II sequences, or one Class I and one Class II sequence.

In another embodiment, the peptide epitopes are in the form of a concatemeric cancer antigen comprised of peptide epitopes. Any number of peptide epitopes may be used. In certain embodiments, the peptide epitopes are in the form of a concatemeric cancer antigen comprised of 5-200 peptide epitopes. In certain embodiments, the peptide epitopes are in the form of a concatemeric cancer antigen comprised of 5-130 peptide epitopes. In some embodiments, the concatemeric cancer antigen comprises one or more of: a) the peptide epitopes (e.g., the 5-200 or 5-130 peptide epitopes) are interspersed by cleavage sensitive sites; and/or b) each peptide epitope is linked directly to one another without a linker; and/or c) each peptide epitope is linked to one or another with a single amino acid linker; and/or d) each peptide epitope is linked to one or another with a short linker; and/or e) each peptide epitope comprises 8-31 amino acids and includes one or more SNP mutations (e.g., a centrally located SNP mutation); and/or f) each peptide epitope comprises 8-31 amino acids and includes a mutation causing a unique expressed peptide sequence; and/or g) the nucleic acids encoding the peptide epitopes are arranged such that the peptide epitopes are ordered to minimize pseudo-epitopes, and/or h) no class II MHC molecules peptide epitopes are present.

It will be appreciated that a concatamer of 2 or more peptides, e.g., 2 or more neoantigens, may create unintended new epitopes (pseudoepitopes) at peptide boundaries. To prevent or eliminate such pseudoepitopes, class I alleles may be scanned for hits across peptide boundaries in a concatamer. In some embodiments, the peptide order within the concatamer is shuffled to reduce or eliminate pseudoepitope formation. In some embodiments, a linker is used between peptides, e.g., a single amino acid linker such as glycine, to reduce or eliminate pseudoepitope formation. In some embodiments, anchor amino acids can be replaced with other amino acids which will reduce or eliminate pseudoepitope formation. In some embodiments, peptides are trimmed at the peptide boundary within the concatamer to reduce or eliminate pseudoepitope formation.

In some embodiments the multiple peptide epitope antigens are arranged and ordered to minimize pseudoepitopes. In some embodiments glycine insertion can be used to disrupt pseudoepitopes. In other embodiments the multiple peptide epitope antigens are a polypeptide that is free of pseudoepitopes. When the cancer antigen epitopes are arranged in a concatemeric structure in a head to tail formation a junction is formed between each of the cancer antigen epitopes. That includes several, i.e., 1-10, amino acids from an epitope on a N-terminus of the peptide and several, i.e., 1-10, amino acids on a C-terminus of an adjacent directly linked epitope. It is important that the junction not be an immunogenic peptide that may produce an immune response. In some embodiments the junction forms a peptide sequence that binds to an HLA protein of a subject for which the personalized cancer vaccine is designed with an IC₅₀ greater than about 50 nM. In other embodiments the junction peptide sequence binds to an HLA protein of a subject with an IC₅₀ greater than about 10 nM, 150 nM, 200 nM, 250 nM, 300 nM, 350 nM, 400 nM, 450 nm, or 500 nM.

### Personalized Cancer Vaccines

In some aspects, the present disclosure provides a nucleic acid cancer vaccine comprising one or more nucleic acids, wherein each of the nucleic acids encodes at least one suitable cancer antigen such as a personalized antigen specific for a cancer subject. A personalized cancer antigen is a tumor-specific antigen, also referred to as a neoantigen, that is present in a tumor of an individual that is not expressed or expressed at low levels in normal non-cancerous tissue of the individual. The antigen may or may not be present in tumors of other individuals.

For instance, the nucleic acid cancer vaccine may include nucleic acids encoding one or more cancer antigens specific for each subject, referred to as neoepitopes. Antigens that are expressed in or by tumor cells are referred to as “tumor associated antigens.” A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes are completely foreign to the body and thus would not produce an immune response against healthy tissue or be masked by the protective components of the immune system. In some embodiments personalized vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations.

In some embodiments the nucleic acid cancer vaccines and vaccination methods described herein may include peptide epitopes or antigens based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

In some embodiments, the nucleic acid cancer vaccines and methods described herein may include peptide epitopes based on cancer/testis (CT) antigens. Cancer/testis antigen expression is limited to male germ cells in healthy adults, but ectopic expression has been observed in tumor cells of multiple types of human cancer. Since male germ cells are devoid of HLA-class I molecules and cannot present antigens to T cells, cancer/testis antigens are generally considered neoantigens when expressed in cancer cells and have the capacity to elicit immune responses that are strictly cancer-specific. Cancer/testis antigens for use with the compositions and methods described herein may be any such cancer/testis antigen known in the field including, but not limited to, MAGEA1, MAGEA2, MAGEA3, MAGEA4, MAGEA5, MAGEA6, MAGEA8, MAGEA9, MAGEA10, MAGEA11, MAGEA12, BAGE, BAGE2, BAGE3, BAGE4, BAGE5, MAGEB1, MAGEB2, MAGEB5, MAGEB6, MAGEB3, MAGEB4, GAGE1, GAGE2A, GAGE3, GAGE4, GAGE5, GAGE6, GAGE7, GAGE8, SSX1, SSX2, SSX2b, SSX3, SSX4, CTAG1B, LAGE-1b, CTAG2, MAGEC1, MAGEC3, SYCP1, BRDT, MAGEC2, SPANXA1, SPANXB1, SPANXC, SPANXD, SPANXN1, SPANXN2, SPANXN3, SPANXN4, SPANXN5, XAGE1D, XAGE1C, XAGE1B, XAGE1, XAGE2, XAGE3, XAGE-3b, XAGE-4/RP11-167P23.2, XAGE5, DDX43, SAGE1, ADAM2, PAGE5, CT16.2, PAGE1, PAGE2, PAGE2B, PAGE3, PAGE4, LIPI, VENTXP1, IL13RA2, TSP50, CTAGE1, CTAGE-2, CTAGE5, SPA17, ACRBP, CSAG1, CSAG2, DSCR8, MMA1b, DDX53, CTCFL, LUZP4, CASC5, TFDP3, JARID1B, LDHC, MORC1, DKKL1, SPO11, CRISP2, FMR1NB, FTHL17, NXF2, TAF7L, TDRD1, TDRD6, TDRD4, TEX15, FATE1, TPTE, CT45A1, CT45A2, CT45A3, CT45A4, CT45A5, CT45A6, HORMAD1, HORMAD2, CT47A1, CT47A2, CT47A3, CT47A4, CT47A5, CT47A6, CT47A7, CT47A8, CT47A9, CT47A10, CT47A11, CT47B1, SLCO6A1, TAG, LEMD1, HSPB9, CCDC110, ZNF165, SPACA3, CXorf48, THEG, ACTL8, NLRP4, COX6B2, LOC348120, CCDC33, LOC196993, PASD1, LOC647107, TULP2, CT66/AA884595, PRSS54, RBM46, CT69/BC040308, CT70/BI818097, SPINLW1, TSSK6, ADAM29, CCDC36, LOC440934, SYCE1, CPXCR1, TSPY3, TSGA10, HIWI, MIWI, PIWI, PIWIL2, ARMC3, AKAP3, Cxorf61, PBK, C21orf99, OIPS, CEP290, CABYR, SPAG9, MPHOSPH1, ROPN1, PLAC1, CALR3, PRM1, PRM2, CAGE1, TTK, LY6K, IMP-3, AKAP4, DPPA2, KIAA0100, DCAF12, SEMG1, POTED, POTEE, POTEA, POTEG, POTEB, POTEC, POTEH, GOLGAGL2 FA, CDCA1, PEPP2, OTOA, CCDC62, GPATCH2, CEP55, FAM46D, TEX14, CTNNA2, FAM133A, LOC130576, ANKRD45, ELOVL4, IGSF11, TMEFF1, TMEFF2, ARX, SPEF2, GPAT2, TMEM108, NOL4, PTPN20A, SPAG4, MAEL, RQCD1, PRAME, TEX101, SPATA19, ODF1, ODF2, ODF3, ODF4, ATAD2, ZNF645, MCAK, SPAG1, SPAG6, SPAG8, SPAG17, FBXO39, RGS22, cyclin A1, C15orf60, CCDC83, TEKT5, NR6A1, TMPRSS12, TPPP2, PRSS55, DMRT1, EDAG, NDR, DNAJB8, CSAG3B, CTAG1A, GAGE12B, GAGE12C, GAGE12D, GAGE12E, GAGE12F, GAGE12G, GAGE12H, GAGE12I, GAGE12J, GAGE13, LOC728137, MAGEA2B, MAGEA9B/LOC728269, NXF2B, SPANXA2, SPANXB2, SPANXE, SSX4B, SSX5, SSX6, SSX7, SSX9, TSPY1D, TSPY1E, TSPY1F, TSPY1G, TSPY1H, TSPY1I, TSPY2, XAGE1E, XAGE2B/CTD-2267G17.3, and/or variants thereof.

In some embodiments, the nucleic acid cancer vaccines may further include one or more nucleic acids encoding for one or more non-mutated tumor antigens. In some embodiments, the nucleic acid cancer vaccines may further include one or more nucleic acids encoding for one or more mutated tumor antigens.

Many tumor antigens are known in the art. Cancer or tumor antigens (e.g., traditional cancer antigens) for use with the compositions and methods described herein may be any such cancer or tumor antigens known in the field. In some embodiments, the cancer or tumor antigen (e.g., the traditional cancer antigen) is one of the following antigens: CD2, CD19, CD20, CD22, CD27, CD33, CD37, CD38, CD40, CD44, CD47, CD52, CD56, CD70, CD79, CD137, 4-IBB, 5T4, AGS-5, AGS-16, Angiopoietin 2, B2M, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin av, Integrin αvβ, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and/or variants thereof.

Epitopes can be identified using a free or commercial database (Lonza Epibase, antitope for example). Such tools are useful for predicting the most immunogenic epitopes within a target antigen protein. The selected peptides may then be synthesized and screened in human HLA panels, and the most immunogenic sequences are used to construct the nucleic acids encoding the peptide epitope(s). One strategy for mapping epitopes of Cytotoxic T-Cells based on generating equimolar mixtures of the four C-terminal peptides for each nominal 11-mer across a protein. This strategy would produce a library antigen containing all the possible active CTL epitopes.

The neoepitopes may be designed to optimally bind to MHC in order to promote a robust immune response. In some embodiments each peptide epitope comprises an antigenic region and a MHC stabilizing region. An MHC stabilizing region is a sequence which stabilizes the peptide in the MHC.

All of the MHC stabilizing regions within the epitopes may be the same or they may be different. The MHC stabilizing regions may be at the N terminal portion of the peptide or the C terminal portion of the peptide. Alternatively the MHC stabilizing regions may be in the central region of the peptide.

The MHC stabilizing region may be 5-10, 5-15, 8-10, 1-5, 3-7, or 3-8 amino acids in length. In yet other embodiments the antigenic region is 5-100 amino acids in length. The peptides interact with the molecules of MHC class I by competitive affinity binding within the endoplasmic reticulum, before they are presented on the cell surface. The affinity of an individual peptide is directly linked to its amino acid sequence and the presence of specific binding motifs in defined positions within the amino acid sequence. The peptide being presented in the MHC is held by the floor of the peptide-binding groove, in the central region of the α1/α2 heterodimer (a molecule composed of two nonidentical subunits). The sequence of residues of the peptide-binding groove's floor determines which particular peptide residues it binds.

Optimal binding regions may be identified by a computer assisted comparison of the affinity of a binding site (MHC pocket) for a particular amino acid at each amino acid in the binding site for each of the target epitopes to identify an ideal binder for all of the examined antigens. The MHC stabilization regions of the epitopes may be identified using amino acid prediction matrices of data points for a binding site. An amino acid prediction matrix is a table having a first and a second axis defining data points. Prediction matrices can be generated as shown in Singh, H. and Raghava, G. P. S. (2001), “ProPred: prediction of HLA-DR binding sites.” Bioinformatics, 17(12), 1236-37). In some embodiments, the prediction matrix is based on evolutionary conservation, in another embodiment, the prediction matrix uses physiochemical similarity to examine how similar a somatic amino acid is to the germline amino acid (e.g., Kim et al., J Immunol. 2017: 3360-3368). The similarity of the somatic amino acid to the germline amino acid approximates how a mutation affects binding (e.g., T cell receptor recognition). In some embodiments, less similarity is indicative of improved binding (e.g., T cell receptor recognition).

In some embodiments the MHC stabilizing region is designed based on the subject's particular MHC. In that way the MHC stabilizing region can be optimized for each patient.

The neoepitopes selected for inclusion in the cancer vaccine (e.g., nucleic acid cancer vaccine) will typically be high affinity binding peptides. In some aspect the neoepitope binds an HLA protein with greater affinity than a wild-type peptide. The neoepitope has an IC₅₀ of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC₅₀<50 nM, are generally considered medium to high affinity binding peptides and will be selected for testing their affinity empirically using biochemical assays of HLA-binding. Finally, it will be determined whether the human immune system can mount effective immune responses against these mutated tumor antigens and thus effectively kill tumor but not normal cells.

In some embodiments, the neoepitopes are 13 residues or less in length and may consist of between about 8 and about 11 residues, particularly 9 or 10 residues. In other embodiments the neoepitopes may be designed to be longer. For instance, the neoepitopes may have extensions of 2-5 amino acids toward the N- and C-terminus of each corresponding gene product. The use of a longer peptide may allow endogenous processing by patient cells and may lead to more effective antigen presentation and induction of T cell responses.

Neoepitopes having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T cell or B cell. For instance, the neoepitopes may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Be, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

The neoepitopes can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell or B cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

The neoepitopes may also comprise isosteres of two or more residues in the neoepitopes. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the alpha-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

The consideration of immunogenicity is an important component in the selection of optimal neoepitopes for inclusion in a vaccine. As a set of non-limiting examples, immunogenicity may be assessed by analyzing the MHC binding capacity of a neoepitope, HLA promiscuity, mutation position, predicted T cell reactivity, actual T cell reactivity, structure leading to particular conformations and resultant solvent exposure, and representation of specific amino acids.

One important aspect of a neoepitope included in a vaccine is a lack of self-reactivity. The putative neoepitopes may be screened to confirm that the epitope is restricted to tumor tissue, for instance, arising as a result of genetic change within malignant cells. Ideally, the epitope should not be present in normal tissue of the patient and thus, self-similar epitopes are filtered out of the dataset. A personalized coding genome may be used as a reference for comparison of neoantigen candidates to determine lack of self-reactivity. In some embodiments, a personalized coding genome is generated from an individualized transcriptome and/or exome.

### Checkpoint Inhibitors

In other aspects the disclosure provides anti-cancer immunotherapies, such as checkpoint inhibitors, for use in combination with the cancer vaccines. Immune checkpoint modulators include both stimulatory checkpoint molecules and inhibitory checkpoint molecules (e.g., an anti-CTLA4 and/or an anti-PD1 antibody).

Stimulatory checkpoint inhibitors function by promoting the checkpoint process. Several stimulatory checkpoint molecules are members of the tumor necrosis factor (TNF) receptor superfamily (e.g., CD27, CD40, OX40, GITR, or CD137), while others belong to the B7-CD28 superfamily (e.g., CD28 or ICOS0. OX40 (CD134), is involved in the expansion of effector and memory T cells. Anti-OX40 monoclonal antibodies have been shown to be effective in treating advanced cancer. MEDI0562 is a humanized OX40 agonist. GITR, Glucocorticoid-Induced TNFR family Related gene, is involved in T cell expansion. Several antibodies to GITR have been shown to promote an anti-tumor responses. ICOS, Inducible T-cell costimulator, is important in T cell effector function. CD27 supports antigen-specific expansion of naïve T cells and is involved in the generation of T and B cell memory. Several agonistic anti-CD27 antibodies are in development. CD122 is the Interleukin-2 receptor beta sub-unit. NKTR-214 is a CD122-biased immune-stimulatory cytokine.

Inhibitory checkpoint molecules include, but are not limited to: PD-1, TIM-3, VISTA, A2AR, B7-H3, B7-H4, BTLA, CTLA-4, IDO, KIR and LAGS. CTLA-4, PD-1, and ligands thereof are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response. Pembrolizumab (formerly MK-3475 and lambrolizumab, trade name KETRUDA) is a human antibody used in cancer immunotherapy and targets the PD-1 receptor.

The checkpoint inhibitor is a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101). In yet other embodiments the checkpoint inhibitor is Pembrolizumab.

Pembrolizumab is a potent humanized immunoglobulin G4 monoclonal antibody with high specificity of binding to the PD-1 receptor, thus inhibiting its interaction with PD-L1 and programmed cell death 1 ligand 2. Based on preclinical in vitro data, pembrolizumab has high affinity and potent receptor blocking activity for PD-1. Pembrolizumab has an acceptable preclinical safety profile and is in clinical development as an IV immunotherapy for advanced malignancies. KEYTRUDA™ (pembrolizumab) is approved for the treatment of patients across a number of indications. Pembrolizumab is approved for use in several cancer types, and is under investigation in several phases of clinical development for many more. Despite much progress in the field of immune-oncology therapeutics, not all subjects respond to pembrolizumab therapy, most responses are not complete, and it is only approved for use in limited tumor types. Combining pembrolizumab with mRNA cancer vaccine may allow more subjects to derive greater clinical benefit than with pembrolizumab monotherapy.

The dose of pembrolizumab in some embodiments is 200 mg administered every 3 weeks. The dose recently approved in the United States for treatment of cutaneous melanoma subjects is 2 mg/kg every 3 weeks. It has been concluded that a dose of 200 mg consistently across multiple tumor types is similar to 2 mg/kg.

In some embodiments the cancer therapeutic agents, including the checkpoint inhibitors, are delivered in the form of mRNA encoding the cancer therapeutic agents. In other embodiments the checkpoint inhibitors are delivered in the form of peptides.

### Methods for Preparation

In other aspects the disclosure provides a method for preparing a cancer vaccine, comprising: a) identifying between 5-130 personalized cancer antigens for a patient; b) determining the anti-tumor efficacy of at least two peptide epitopes for each of the 5-130 personalized cancer antigens; and c) preparing a cancer vaccine in which the total anti-cancer efficacy of the cancer vaccine is maximized (e.g., the predicted total anti-cancer efficacy of the cancer vaccine is maximized) for a given total length of the cancer vaccine.

Methods for generating cancer vaccines according to the disclosure may involve identification of mutations using techniques such as deep nucleic acid or protein sequencing methods as described herein of tissue samples. In some embodiments an initial identification of mutations in a subject's (e.g., a patient's) transcriptome is performed. The data from the subject's (e.g., the patient's) transcriptome is compared with sequence information from the subject's (e.g., the patient's) exome in order to identify patient specific and tumor specific mutations that are expressed. The comparison produces a dataset of putative neoepitopes, referred to as a mutanome. The mutanome may include approximately 100-10,000 candidate mutations per patient. In some embodiments an mRNA neoantigen vaccine is designed and manufactured. The patient is then treated with the vaccine. In certain embodiments, such a neoantigen-containing vaccine may be a polycistronic vaccine including multiple neoepitopes or one or more single RNA vaccines or a combination thereof.

In some embodiments the entire method from the initiation of the mutation identification process to the start of patient treatment is achieved in less than 2 months. In other embodiments the whole process is achieved in 7 weeks or less, 6 weeks or less, 5 weeks or less, 4 weeks or less, 3 weeks or less, 2 weeks or less or less than 1 week. In some embodiments the whole method is performed in less than 30 days.

In a personalized cancer vaccine, the subject specific cancer antigens may be identified in a sample of a patient. The term “biological sample” refers to a sample that contains biological materials such as a DNA, a RNA and a protein. In some embodiments, the biological sample may suitably comprise a bodily fluid from a subject. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In some embodiments, the sample may be a tissue sample or a tumor sample. For instance, a sample of one or more tumor cells may be examined for the presence of subject specific cancer antigens.

Once an mRNA vaccine is synthesized, it is administered to the patient. In some embodiments the vaccine is administered on a schedule for up to two months, up to three months, up to four month, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments the schedule is weekly for the first 3 weeks and then monthly thereafter.

At any point in the treatment the patient may be examined to determine whether the mutations in the vaccine are still appropriate. Based on that analysis the vaccine may be adjusted or reconfigured to include one or more different mutations or to remove one or more mutations. It has been recognized and appreciated that, by analyzing certain properties of cancer associated mutations, optimal neoepitopes may be assessed and/or selected for inclusion in a cancer vaccine. A property of a neoepitope or set of neoepitopes may include, for instance, an assessment of gene or transcript-level expression in patient RNA-seq or other nucleic acid analysis, tissue-specific expression in available databases, known oncogenes/tumor suppressors, variant call confidence score, RNA-seq allele-specific expression, conservative vs. non-conservative AA substitution, position of point mutation (Centering Score for increased TCR engagement), position of point mutation (Anchoring Score for differential HLA binding), Selfness: <100% core epitope homology with patient WES data, HLA-A and -B IC₅₀ for 8mers-11mers, HLA-DRB1 IC₅₀ for 15mers-20mers, promiscuity Score (i.e., number of patient HLAs predicted to bind), HLA-C IC₅₀ for 8mers-11mers, HLA-DRB3-5 IC₅₀ for 15mers-20mers, HLA-DQB1/A1 IC₅₀ for 15mers-20mers, HLA-DPB1/A1 IC₅₀ for 15mers-20mers, Class I vs Class II proportion, Diversity of patient HLA-A, —B and DRB1 allotypes covered, proportion of point mutation vs complex epitopes (e.g., frameshifts), and/or pseudo-epitope HLA binding scores.

In some embodiments, the properties of cancer associated mutations used to identify optimal neoepitopes are properties related to the type of mutation, abundance of mutation in patient sample, immunogenicity, lack of self-reactivity, and nature of peptide composition. The type of mutation should be determined and considered as a factor in determining whether a putative epitope should be included in a vaccine. The type of mutation may vary. In some instances it may be desirable to include multiple different types of mutations in a single vaccine. In other instances a single type of mutation may be more desirable. A value for each particular mutation can be weighted and calculated. In some embodiments, a particular mutation is a single nucleotide polymorphism (SNP). In some embodiments, a particular mutation is a complex variant, for example, a peptide sequence resulting from intron retention, complex splicing events, or insertion/deletion mutations changing the reading frame of a sequence.

The abundance of the mutation in a patient sample may also be scored and factored into the decision of whether a putative epitope should be included in a vaccine. Highly abundant mutations may promote a more robust immune response.

In some embodiments, the personalized mRNA cancer vaccines described herein may be used for treatment of cancer. As one non-limiting example, the disclosure provides methods for treating a patient having cancer, comprising: a) analyzing a sample derived from the patient is in order to identify one or more personalized cancer antigens; b) determining the anti-tumor efficacy of at least two peptide epitopes for each of the identified personalized cancer antigens; c) preparing a cancer vaccine in which the total anti-cancer efficacy of the cancer vaccine is maximized (e.g., the predicted total anti-cancer efficacy of the cancer vaccine is maximized) for a given total length of the cancer vaccine; and d) administering the cancer vaccine to the patient.

Cancer vaccines (e.g., nucleic acid cancer vaccines) may be administered prophylactically or therapeutically as part of an active immunization scheme to healthy individuals or early in cancer or late stage and/or metastatic cancer. In one embodiment, the effective amount of the cancer vaccine (e.g., nucleic acid cancer vaccines) provided to a cell, a tissue or a subject may be enough for immune activation, and in particular antigen specific immune activation.

In some embodiments, the cancer vaccine (e.g., nucleic acid cancer vaccine) may be administered with an anti-cancer therapeutic agent. The cancer vaccine (e.g., nucleic acid cancer vaccine) and anti-cancer therapeutic can be combined to enhance immune therapeutic responses even further. The cancer vaccine (e.g., nucleic acid cancer vaccines) and other therapeutic agent may be administered simultaneously or sequentially. When the other therapeutic agents are administered simultaneously they can be administered in the same or separate formulations, but are administered at the same time. The other therapeutic agents are administered sequentially with one another and with the cancer vaccine (e.g., nucleic acid cancer vaccine), when the administration of the other therapeutic agents and the cancer vaccine (e.g., nucleic acid cancer vaccine) is temporally separated. The separation in time between administrations of these compounds may be a matter of minutes or it may be longer, e.g., hours, days, weeks, months. Other therapeutic agents include but are not limited to anti-cancer therapeutic, adjuvants, cytokines, antibodies, antigens, etc. Examples of anti-cancer therapeutics include, but are not limited to, DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), hormone therapy (e.g., tamoxifen, flutamide), and gene-targeted therapies, such as protein-tyrosine kinase inhibitors (e.g. imatinib; the EGFR kinase inhibitor, erlotinib). In some embodiments, the anti-cancer therapeutic is pembrolizumab.

In some embodiments, the progression of the cancer can be monitored to identify changes in the expressed antigens. Thus, in some embodiments the method also involves at least one month after the administration of a cancer mRNA vaccine, identifying at least 2 cancer antigens from a sample of the subject to produce a second set of cancer antigens, and administering to the subject a mRNA vaccine having an open reading frame encoding the second set of cancer antigens to the subject. The mRNA vaccine having an open reading frame encoding second set of antigens, in some embodiments, is administered to the subject 2 months, 3 months, 4 months, 5 months, 6 months, 8 months, 10 months, or 1 year after the mRNA vaccine having an open reading frame encoding the first set of cancer antigens. In other embodiments the mRNA vaccine having an open reading frame encoding second set of antigens is administered to the subject 1½, 2, 2½, 3, 3½, 4, 4½, or 5 years after the mRNA vaccine having an open reading frame encoding the first set of cancer antigens.

### Hotspot Mutations as Neoantigens

In population analyses of cancer, certain mutations occur in a higher percentage of patients than would be expected by chance. These “recurrent” or “hotspot” mutations have often been shown to have a “driver” role in the tumor, producing some change in the cancer cell function that is important to tumor initiation, maintenance, or metastasis, and is therefore selected for in the evolution of the tumor. In addition to their importance in tumor biology and therapy, recurrent mutations provide the opportunity for precision medicine, in which the patient population is stratified into groups more likely to respond to a particular therapy, including but not limited to targeting the mutated protein itself.

Therefore, in some embodiments, the cancer vaccine further comprises one or more cancer hotspot neoepitopes in addition the personalized cancer epitopes. In some embodiments, one or more cancer hotspot neoepitopes are cancer hotspot antigens. In some embodiments, cancer hotspot mutations that occur over a threshold prevalence in an indication of interest are included in the vaccine. The threshold prevalence, in some embodiments, is greater than 2%, 3%, 4%, 5%, 6%, 7%, 8%, 9%, or 10%.

Indications of interest include, but are not limited to bladder urothelial carcinoma (BLCA), colon adenocarcinoma (COAD), esophageal carcinoma (ESCA), hepatocellular carcinoma (HCC), head and neck squamous cell carcinoma (HNSC), lung adenocarcinoma (LUAD), pancreatic adenocarcinoma (PAAD), prostate adenocarcinoma (PRAD), rectum adenocarcinoma (READ), small cell lung cancer (SCLC), skin cutaneous melanoma (SKCM), serous ovarian cancer (SOC), stomach adenocarcinoma (STAD), and uterine endometrial cancer (UEC). Exemplary mutations are provided in the table below.

| Gene | Mutated position |
|---|---|
| KRAS | G12, G13 |
| NRAS | Q61 |
| BRAF | V600 |
| PIK3CA | R88, E545, H1047 |
| TP53 | R175, R282 |
| EGFR | L858 |
| FGFR3 | S249 |
| ERBB2 | S310 |
| PTEN | R130 |
| BCOR | N1459 |

Much effort and research on recurrent mutations has focused on non-synonymous (or “missense”) single nucleotide variants (SNVs), but population analyses have revealed that a variety of more complex (non-SNV) variant classifications, such as synonymous (or “silent”), splice site, multi-nucleotide variants, insertions, and deletions, can also occur at high frequencies.

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. Surprisingly, a small subset of p53 loci do, however, exhibit a “hotspot” pattern, in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery. Mutation of a splicing motif can alter the final mRNA sequence even if no change to the local amino acid sequence is predicted (i.e., for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no PTC is produced), it can escape depletion by NMD and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

Mutations are typically obtained from a patient's DNA sequencing data to derive neo-epitopes for prior art peptide vaccines. mRNA expression, however, is a more direct measurement of the global space of possible neo-epitopes. For example, some tumor-specific neo-epitopes may arise from splicing changes, insertions/deletions (InDels) resulting in frameshifts, alternative promoters, or epigenetic modifications that are not easily identified using only the exome sequencing data. In some aspects, the neoantigens from InDels are enriched for predicted high-affinity binders versus nsSNVs. Such neoantigens may be immunogenic. For example, frameshift InDels have been found to be significantly associated with checkpoint inhibitor responses across three melanoma cohorts.

In some aspects, the invention involves a method for identifying patient specific complex mutations and formulating these mutations into effective personalized cancer vaccines (e.g., nucleic acid cancer vaccines). The methods involve the use of short read RNA-Seq. A major challenge inherent to using short reads for RNA-seq is the fact that multiple mRNA transcript isoforms can be obtained from the same genomic locus, due to alternative splicing and other mechanisms. Due to the sequencing reads being much shorter than the full-length mRNA transcript, it becomes difficult to map a set of reads back to the correct corresponding isoform within a known gene annotation model. As a result, complex variants that diverge from the known gene annotations (as are common in cancer) can be difficult to discover by standard approaches. However, short peptides may be identified rather than the exact exon composition of the full-length transcript. The methods for identifying short peptides that will be representative of these complex mutations involve a short k-mer counting approach to neo-epitope prediction of complex variants.

### Biomarkers

Next-generation sequencing was used to analyze patient data (pre-treatment biopsies) to understand and characterize the mutation landscape, expression level of key genes, and tumor microenvironment of the patients prior to treatment. By correlating to neoantigen-specific T cell responses post-vaccination, the data provided important biomarkers to assist in patient/therapeutic selection for personalized neoantigen cancer vaccines.

The biomarkers include microsatellite instability (MSI) value, tumor mutational burden (TMB) or T-cell inflamed gene expression profile (GEP). The GEP score includes at least 18 genes including: CXCR6, TIGIT, CD27, CD274 (PD-L1), PDCD1LG2 (PD-L2), LAG3, NKG7, PSMB10, CMKLR1, CD8A, IDO1, CCL5, CXCL9, HLA-DQA1, CD276, HLA-DRB1, STAT1, HLA-E. In some embodiments the GEP includes PD-1, PD-L1 and PD-L2. The subject may be selected for treatment based on a threshold GEP and TMB.

### Nucleic Acids/Polynucleotides

Cancer vaccines (e.g., nucleic acid cancer vaccines), as provided herein, comprise at least one (one or more) nucleic acid having an open reading frame encoding at least one peptide epitope. The term “nucleic acid,” in its broadest sense, includes any compound and/or substance that comprises a polymer of nucleotides. These polymers are also referred to as polynucleotides.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

As a non-limiting example, when a DNA nucleic acid cancer vaccine as described herein is delivered to a cell, the DNA is transcribed into RNA, and the RNA will be processed into a polypeptide by the intracellular machinery which can then process the polypeptide into immunosensitive fragments capable of stimulating an immune response against a tumor or population of cancerous cells. As a non-limiting example, when an RNA (e.g., mRNA) nucleic acid cancer vaccine as described herein is delivered to a cell, the RNA (e.g., mRNA) will be processed into a polypeptide by the intracellular machinery which can then process the polypeptide into immunosensitive fragments capable of stimulating an immune response against a tumor or population of cancerous cells.

In some embodiments, nucleic acids of the present disclosure function as messenger RNA (mRNA). “Messenger RNA” (mRNA) refers to any nucleic acid that encodes a (at least one) polypeptide (a naturally-occurring, non-naturally-occurring, or modified polymer of amino acids) and can be translated to produce the encoded polypeptide in vitro, in vivo, in situ or ex vivo.

The basic components of an mRNA molecule typically include at least one coding region, a 5′ untranslated region (UTR), a 3′ UTR, a 5′ cap and a poly-A tail. Nucleic acids of the present disclosure may function as mRNA but can be distinguished from wild-type mRNA in their functional and/or structural design features which serve to overcome existing problems of effective polypeptide expression using nucleic-acid based therapeutics.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 90% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 85% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 80% sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares less than 75% sequence identity with a naturally-occurring or wild-type sequence (e.g., a naturally-occurring or wild-type mRNA sequence encoding a polypeptide or protein of interest (e.g., an antigenic protein or polypeptide).

In some embodiments, a codon optimized sequence shares between 65% and 85% (e.g., between about 67% and about 85% or between about 67% and about 80%) sequence identity with a naturally-occurring or wild-type sequence (e.g., a naturally-occurring or wild-type mRNA sequence encoding a polypeptide or protein of interest (e.g., an antigenic protein or polypeptide). In some embodiments, a codon optimized sequence shares between 65% and 75% or about 80% sequence identity with a naturally-occurring or wild-type sequence (e.g., a naturally-occurring or wild-type mRNA sequence encoding a polypeptide or protein of interest (e.g., an antigenic protein or polypeptide).

In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemical Modifications

### Modified Nucleotide Sequences Encoding Epitope Antigen Polypeptides

In some embodiments, the nucleic acid cancer vaccine of the invention comprises one or more chemically modified nucleobases. The invention includes modified polynucleotides comprising a polynucleotide described herein (e.g., a nucleic acid comprising a nucleotide sequence encoding one or more cancer peptide epitopes). The modified nucleic acids can be chemically modified and/or structurally modified. When the nucleic acids of the present invention are chemically and/or structurally modified the polynucleotides can be referred to as “modified nucleic acids.”

The present disclosure provides for modified nucleosides and nucleotides of a nucleic acid (e.g., RNA polynucleotides, such as mRNA polynucleotides) encoding one or more cancer peptide epitopes. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can by synthesized by any useful method, such as, for example, chemically, enzymatically, or recombinantly, to include one or more modified or non-natural nucleosides. Nucleic acids can comprise a region or regions of linked nucleosides. Such regions can have variable backbone linkages. The linkages can be standard phosphodiester linkages, in which case the polynucleotides would comprise regions of nucleotides.

The modified nucleic acids disclosed herein can comprise various distinct modifications. In some embodiments, the modified polynucleotides contain one, two, or more (optionally different) nucleoside or nucleotide modifications. In some embodiments, a modified polynucleotide introduced to a cell can exhibit one or more desirable properties such as, e.g., improved protein expression, reduced immunogenicity, or reduced degradation in the cell, as compared to an unmodified polynucleotide.

In some embodiments, a nucleic acid disclosed herein (e.g., a nucleic acid encoding one or more peptide epitopes) is structurally modified. As used herein, a “structural” modification is one in which two or more linked nucleosides are inserted, deleted, duplicated, inverted, or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G.” The same polynucleotide can be structurally modified from “ATCG” to “ATCCCG.” Here, the dinucleotide “CC” has been inserted, resulting in a structural modification to the nucleic acid.

In some embodiments, the nucleic acids of the instant disclosure are chemically modified. As used herein in reference to a nucleic acid, the terms “chemical modification” or, as appropriate, “chemically modified” refer to modification with respect to adenosine (A), guanosine (G), uridine (U), or cytidine (C) ribo- or deoxyribonucleosides in one or more of their position, pattern, percentage, or population. Generally, herein, these terms are not intended to refer to the ribonucleotide modifications in naturally occurring 5′-terminal mRNA cap moieties.

In some embodiments, the nucleic acids of the instant disclosure can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine, or uracil. Any combination of base/sugar or linker can be incorporated into polynucleotides of the present disclosure.

The skilled artisan will appreciate that, except where otherwise noted, nucleic acid sequences set forth in the instant application will recite “T”s in a representative DNA sequence but where the sequence represents RNA, the “T”s would be substituted for “U”s.

Cancer vaccines of the present disclosure comprise, in some embodiments, at least one nucleic acid (e.g., RNA) having an open reading frame encoding at least one (e.g., 5-200 or 5-130) peptide epitope(s), wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein for this purpose.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., DNA nucleic acids or RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U, or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90%, or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some embodiments, the nucleic acid can include any useful linker between the nucleosides. Such linkers, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

The modified nucleosides and nucleotides (e.g., building block molecules), which can be incorporated into a nucleic acid (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C6-10 aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C1-6 alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl; aminoalkoxy; amino; and amino acid.

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are described in, for example, International Patent Publication Nos. WO2013052523 and WO2014093924, the contents of each of which are incorporated herein by reference in their entireties for this purpose.

The nucleic acids of the disclosure (e.g., a nucleic acid encoding one or more peptide epitopes or a functional fragment or variant thereof) can include a combination of modifications to the sugar, the nucleobase, and/or the internucleoside linkage. These combinations can include any one or more modifications described herein.

The nucleic acid cancer vaccines disclosed herein are compositions, including pharmaceutical compositions. The disclosure also encompasses methods for the selection, design, preparation, manufacture, formulation, and/or use of nucleic acid cancer vaccines as provided herein. Also provided are systems (e.g., computerized systems), processes, devices and kits for the selection, design, and/or utilization of the nucleic acid cancer vaccines described herein.

In Vitro Transcription of RNA (e.g., mRNA)

Cancer vaccines of the present disclosure may comprise at least one nucleic acid (e.g., an RNA polynucleotide, such as an mRNA (message RNA) or an mmRNA (modified mRNA)). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In some embodiments, a nucleic acid includes 15 to 3,000 nucleotides. For example, a polynucleotide may include 15 to 50, 15 to 100, 15 to 200, 15 to 300, 15 to 400, 15 to 500, 15 to 600, 15 to 700, 15 to 800, 15 to 900, 15 to 1000, 15 to 1200, 15 to 1400, 15 to 1500, 15 to 1800, 15 to 2000, 15 to 2500, 15 to 3000, 50 to 100, 50 to 200, 50 to 300, 50 to 400, 50 to 500, 50 to 600, 50 to 700, 50 to 800, 50 to 900, 50 to 1000, 50 to 1200, 50 to 1400, 50 to 1500, 50 to 1800, 50 to 2000, 50 to 2500, 50 to 3000, 100 to 200, 100 to 300, 100 to 400, 100 to 500, 100 to 600, 100 to 700, 100 to 800, 100 to 900, 100 to 1000, 100 to 1200, 100 to 1400, 100 to 1500, 100 to 1800, 100 to 2000, 100 to 2500, 100 to 3000, 200 to 300, 200 to 400, 200 to 500, 200 to 600, 200 to 700, 200, to 800, 200 to 900, 200 to 1000, 200 to 1500, 200 to 3000, 500 to 1000, 500 to 1500, 500 to 2000, 500 to 2500, 500 to 3000, 1000 to 1500, 1000 to 2000, 1000 to 2500, 1000 to 3000, 1500 to 3000, 2500 to 3000, or 2000 to 3000 nucleotides).

In other aspects, the disclosure relates to a method for preparing a nucleic acid cancer vaccine (e.g., an mRNA cancer vaccine) by IVT methods. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities). See Beckert et al., Synthesis of RNA by in vitro transcription, Methods Mol Biol. 703:29-41(2011); Rio et al. RNA: A Laboratory Manual. Cold Spring Harbor: Cold Spring Harbor Laboratory Press, 2011, 205-220; Cooper, Geoffery M. The Cell: A Molecular Approach. 4th ed. Washington D.C.: ASM Press, 2007. 262-299, each of which is herein incorporated by reference for this purpose. Generally, IVT utilizes a DNA template featuring a promoter sequence upstream of a sequence of interest. The promoter sequence is most commonly of bacteriophage origin (e.g., the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that nucleic acid cancer vaccines (e.g., mRNA cancer vaccines) of the present disclosure, e.g., mRNAs encoding the cancer antigen, may be made using any appropriate synthesis method. For example, in some embodiments, mRNA vaccines of the present disclosure are made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide, T3 promoter complementary oligonucleotide, or SP6 promoter complementary oligonucleotide), followed by IVT using an RNA polymerase corresponding to the promoter sequence, e.g., aT7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more peptide epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more peptide epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles, e.g., 3 to 10 cycles. In some embodiments, a double-stranded DNA template is synthesized wholly or in part by chemical synthesis methods. The double-stranded DNA template can be subjected to in vitro transcription as described herein.

In another aspect, nucleic acid cancer vaccines of the present disclosure comprising, e.g., mRNAs encoding the peptide epitopes, may be made using two DNA strands that are complementary across an overlapping portion of their sequence, leaving single-stranded overhangs (i.e., sticky ends) when the complementary portions are annealed. These single-stranded overhangs can be made double-stranded by extending using the other strand as a template, thereby generating double-stranded DNA. In some cases, this primer extension method can permit larger ORFs to be incorporated into the template DNA sequence, e.g., as compared to sizes incorporated into the template DNA sequences obtained by top strand DNA synthesis methods. In the primer extension method, a portion of the 3F-end of a first strand (in the 5′-3′ direction) is complementary to a portion the 3′-end of a second strand (in the 3′-5′ direction). In some such embodiments, the single first strand DNA may include a sequence of a promoter (e.g., T7, T3, or SP6), optionally a 5′-UTR, and some or all of an ORF (e.g., a portion of the 5′-end of the ORF). In some embodiments, the single second strand DNA may include complementary sequences for some or all of an ORF (e.g., a portion complementary to the 3′-end of the ORF), and optionally a 3′-UTR, a stop sequence, and/or a poly(A) tail. Methods of making RNA using two synthetic DNA strands may include annealing the two strands with overlapping complementary portions, followed by primer extension using one or more PCR-like cycles to extend the strands to generate a double-stranded DNA template. In some embodiments, the number of PCR-like cycles ranges from 1 to 20 cycles, e.g., 3 to 10 cycles. Such double-stranded DNA can be subjected to in vitro transcription as described herein.

In another aspect, nucleic acid vaccines of the present disclosure comprising, e.g., mRNAs encoding the peptide epitopes, may be made using synthetic double-stranded linear DNA molecules, such as gBlocks® (Integrated DNA Technologies, Coralville, Iowa), as the double-stranded DNA template. An advantage to such synthetic double-stranded linear DNA molecules is that they provide a longer template from which to generate mRNAs. For example, gBlocks® can range in size from 45-1000 (e.g., 125-750 nucleotides). In some embodiments, a synthetic double-stranded linear DNA template includes a full length 5′-UTR, a full length 3′-UTR, or both. A full length 5′-UTR may be up to 100 nucleotides in length, e.g., about 40-60 nucleotides. A full length 3′-UTR may be up to 300 nucleotides in length, e.g., about 100-150 nucleotides.

To facilitate generation of longer constructs, two or more double-stranded linear DNA molecules and/or gene fragments that are designed with overlapping sequences on the 3′ strands may be assembled together using methods known in art. For example, the Gibson Assembly™ Method (Synthetic Genomics, Inc., La Jolla, Calif.) may be performed with the use of a mesophilic exonuclease that cleaves bases from the 5′-end of the double-stranded DNA fragments, followed by annealing of the newly formed complementary single-stranded 3′-ends, polymerase-dependent extension to fill in any single-stranded gaps, and finally, covalent joining of the DNA segments by a DNA ligase.

In another aspect, nucleic acid cancer vaccines of the present disclosure comprising, e.g., mRNAs encoding the peptide epitopes, may be made using chemical synthesis of the RNA. Methods, for instance, involve annealing a first polynucleotide comprising an open reading frame encoding the polypeptide and a second polynucleotide comprising a 5′-UTR to a complementary polynucleotide conjugated to a solid support. The 3′-terminus of the second polynucleotide is then ligated to the 5′-terminus of the first polynucleotide under suitable conditions. Suitable conditions include the use of a DNA Ligase. The ligation reaction produces a first ligation product. The 5′ terminus of a third polynucleotide comprising a 3′-UTR is then ligated to the 3′-terminus of the first ligation product under suitable conditions. Suitable conditions for the second ligation reaction include an RNA Ligase. A second ligation product is produced in the second ligation reaction. The second ligation product is released from the solid support to produce an mRNA encoding a polypeptide of interest. In some embodiments the mRNA is between 30 and 1000 nucleotides.

An mRNA encoding one or more peptide epitopes may also be prepared by binding a first nucleic acid comprising an open reading frame encoding the nucleic acid to a second nucleic acid comprising 3′-UTR to a complementary nucleic acid conjugated to a solid support. The 5′-terminus of the second nucleic acid is ligated to the 3′-terminus of the first nucleic acid under suitable conditions (including, e.g., a DNA Ligase). The method produces a first ligation product. A third nucleic acid comprising a 5′-UTR is ligated to the first ligation product under suitable conditions (including, e.g., an RNA Ligase, such as T4 RNA) to produce a second ligation product. The second ligation product is released from the solid support to produce an mRNA encoding one or more peptide epitopes.

In some embodiments the first nucleic acid features a 5′-triphosphate and a 3′-OH. In other embodiments the second nucleic acid comprises a 3′-OH. In yet other embodiments, the third nucleic acid comprises a 5′-triphosphate and a 3′-OH. The second nucleic acid may also include a 5′-cap structure. The method may also involve the further step of ligating a fourth nucleic acid comprising a poly-A region at the 3′-terminus of the third nucleic acid. The fourth nucleic acid may comprise a 5′-triphosphate.

The method may or may not comprise reverse phase purification. The method may also include a washing step wherein the solid support is washed to remove unreacted nucleic acids. The solid support may be, for instance, a capture resin. In some embodiments the method involves dT purification.

In accordance with the present disclosure, template DNA encoding the nucleic acid (e.g., mRNA) cancer vaccines of the present disclosure includes an open reading frame (ORF) encoding one or more peptide epitopes. In some embodiments, the template DNA includes an ORF of up to 1000 nucleotides, e.g., about 10-350, 30-300 nucleotides or about 50-250 nucleotides. In some embodiments, the template DNA includes an ORF of about 150 nucleotides. In some embodiments, the template DNA includes an ORF of about 200 nucleotides.

In some embodiments, IVT transcripts are purified from the components of the IVT reaction mixture after the reaction takes place. For example, the crude IVT mix may be treated with RNase-free DNase to digest the original template. The nucleic acid (e.g., mRNA) can be purified using methods known in the art, including but not limited to, precipitation using an organic solvent or column based purification method. Commercial kits are available to purify RNA, e.g., MEGACLEAR™ Kit (Ambion, Austin, Tex.). The nucleic acid (e.g., mRNA) can be quantified using methods known in the art, including but not limited to, commercially available instruments, e.g., NanoDrop. Purified nucleic acids (e.g., mRNAs) can be analyzed, for example, by agarose gel electrophoresis to confirm the nucleic acid is the proper size and/or to confirm that no degradation of the nucleic acid has occurred.

Untranslated Regions (UTRs) Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) of the disclosure comprising an open reading frame (ORF) encoding one or more peptide epitopes further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids of the present disclosure as flanking regions to an ORF. This publication is incorporated by reference herein for this purpose.

Additional exemplary UTRs that may be utilized in the nucleic acids of the present disclosure include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (1743) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (1743) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org/Derrick_Rossi/, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo(dT) sequence for template addition of a poly-A tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an ORF and a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See Chappell et al., PNAS 2004 101:9590-9594, incorporated herein by reference in its entirety for this purpose. The terms “translational enhancer polynucleotide” or “translation enhancer polynucleotide sequence” refer to a nucleic acid that includes one or more of the TEE provided herein and/or known in the art (see, e.g., U.S. Pat. Nos. 6,310,197, 6,849,405, 7,456,273, 7,183,395, US2009/0226470, US2007/0048776, US2011/0124100, US2009/0093049, US2013/0177581, WO2009/075886, WO2007/025008, WO2012/009644, WO2001/055371, WO1999/024595, EP2610341A1, and EP2610340A1; the contents of each of which are incorporated herein by reference in their entirety for this purpose), or their variants, homologs, or functional derivatives. In some embodiments, the nucleic acid of the disclosure comprises one or multiple copies of a TEE. The TEE in a translational enhancer nucleic acid can be organized in one or more sequence segments. A sequence segment can harbor one or more of the TEEs provided herein, with each TEE being present in one or more copies. When multiple sequence segments are present in a translational enhancer nucleic acid, they can be homogenous or heterogeneous. Thus, the multiple sequence segments in a translational enhancer nucleic acid can harbor identical or different types of the TEE provided herein, identical or different number of copies of each of the TEE, and/or identical or different organization of the TEE within each sequence segment. In one embodiment, the nucleic acid of the disclosure comprises a translational enhancer nucleic acid sequence.

In some embodiments, a 5′ UTR and/or 3′ UTR comprising at least one TEE described herein can be incorporated in a monocistronic sequence such as, but not limited to, a vector system or a nucleic acid vector. In some embodiments, a 5′ UTR and/or 3′ UTR of a polynucleotide of the disclosure comprises a TEE or portion thereof described herein. In some embodiments, the TEEs in the 3′ UTR can be the same and/or different from the TEE located in the 5′ UTR. In some embodiments, a 5′ UTR and/or 3′ UTR of a nucleic acid of the disclosure can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55, or more than 60 TEE sequences. In one embodiment, the 5′ UTR of a nucleic acid of the disclosure can include 1-60, 1-55, 1-50, 1-45, 1-40, 1-35, 1-30, 1-25, 1-20, 1-15, 1-10, 9, 8, 7, 6, 5, 4, 3, 2, or 1 TEE sequences. The TEE sequences in the 5′ UTR of the nucleic acid of the disclosure can be the same or different TEE sequences. A combination of different TEE sequences in the 5′ UTR of the nucleic acid of the disclosure can include combinations in which more than one copy of any of the different TEE sequences are incorporated.

In some embodiments, the 5′ UTR and/or 3′ UTR comprises a spacer to separate two TEE sequences. As a non-limiting example, the spacer can be a 15 nucleotide spacer and/or other spacers known in the art (e.g., in multiples of three nucleotides). As another non-limiting example, the 5′ UTR and/or 3′ UTR comprises a TEE sequence-spacer module repeated at least once, at least twice, at least 3 times, at least 4 times, at least 5 times, at least 6 times, at least 7 times, at least 8 times, at least 9 times, at least 10 times, or more than 10 times in the 5′ UTR and/or 3′ UTR, respectively. In some embodiments, the 5′ UTR and/or 3′ UTR comprises a TEE sequence-spacer module repeated 1, 2, 3, 4, 5, 6, 7, 8, 9, or 10 times.

### 3′ UTR and the AU Rich Elements

In certain embodiments, a nucleic acid of the present disclosure (e.g., a nucleic acid encoding a peptide epitope of the disclosure) further comprises a 3′ UTR.

A 3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In one embodiment, the 3′-UTR useful for the disclosure comprises a binding site for regulatory proteins or microRNAs. In some embodiments, the 3′-UTR has a silencer region, which binds to repressor proteins and inhibits the expression of the mRNA. In other embodiments, the 3′-UTR comprises an AU-rich element (AREs). Proteins bind AREs to affect the stability or decay rate of transcripts in a localized manner or affect translation initiation. In other embodiments, the 3′-UTR comprises the sequence AAUAAA that directs addition of several hundred adenine residues called the poly(A) tail to the end of the mRNA transcript.

Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

### Regions Having a 5′ Cap

The nucleic acid cancer vaccine described herein may be an mRNA cancer vaccine comprising one or more mRNA having open reading frames that encode peptide epitopes. Each of these mRNA may have a 5′ Cap.

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule (cap). This 5′-guanylate cap can then be methylated to generate an N7-methyl-guanylate residue (cap-0). The ribose sugars of the terminal and/or anteterminal transcribed nucleotides of the 5′ end of the mRNA can optionally also be 2′-O-methylated (e.g., with a 2′-hydroxy group on the first ribose sugar (cap-1); or with a 2′-hydroxy group on the first two ribose sugars (cap-2)). 5′-decapping through hydrolysis and cleavage of the guanylate cap structure can target a nucleic acid molecule, such as an mRNA molecule, for degradation.

In some embodiments, nucleic acids of the present disclosure (e.g., a nucleic acid encoding a peptide epitope) incorporate a cap moiety.

In some embodiments, nucleic acids of the present disclosure (e.g., a nucleic acid encoding a peptide epitope) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the disclosure.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ 0-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110, the contents of which are herein incorporated by reference in its entirety for this purpose.

In another embodiment, the cap is a cap analog is a N7-(4-chlorophenoxyethyl) substituted dicucleotide form of a cap analog known in the art and/or described herein. Non-limiting examples of a N7-(4-chlorophenoxyethyl) substituted dicucleotide form of a cap analog include a N7-(4-chlorophenoxyethyl)-G(5′)ppp(5′)G and a N7-(4-chlorophenoxyethyl)-m3′-OG(5′)ppp(5′)G cap analog (see, e.g., the various cap analogs and the methods of synthesizing cap analogs described in Kore et al. Bioorganic & Medicinal Chemistry 2013 21:4570-4574; the contents of which are herein incorporated by reference in its entirety for this purpose). In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Nucleic acids of the disclosure (e.g., a nucleic acids encoding peptide antigens) can also be capped post-manufacture (whether through IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., or which outperforms the corresponding endogenous, wild-type, natural or physiological feature in one or more respects. Non-limiting examples of more authentic 5′cap structures are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the cap-1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap-0), 7mG(5′)ppp(5′)NlmpNp (cap-1), and 7mG(5′)-ppp(5′)NlmpN2mp (cap-2).

As a non-limiting example, capping chimeric nucleic acids post-manufacture can be more efficient as nearly 100% of the chimeric nucleic acids can be capped. This is in contrast to ˜80% when a cap analog is linked to a chimeric nucleic acids in the course of an in vitro transcription reaction.

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

In some embodiments, the nucleic acids of the present disclosure (e.g., a nucleic acid encoding peptide epitopes) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a nucleic acid such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240, or 250 residues long. In some embodiments, the poly A tail comprises about 100 nucleotides.

PolyA tails can also be added after the construct is exported from the nucleus.

According to the present disclosure, terminal groups on the poly A tail can be incorporated for stabilization. Polynucleotides of the present disclosure can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety for this purpose).

The nucleic acids of the present disclosure can be designed to encode transcripts with alternative polyA tail structures including histone mRNA. According to Norbury, “[t]erminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety for this purpose.

Unique poly-A tail lengths provide certain advantages to the nucleic acids of the present disclosure. Generally, the length of a poly-A tail, when present, is greater than 30 nucleotides in length. In another embodiment, the poly-A tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the nucleic acid or region thereof includes from about 15 to about 3,000 nucleotides (e.g., from 15 to 50, 15 to 100, 15 to 200, 15 to 300, 15 to 400, 15 to 500, 15 to 600, 15 to 700, 15 to 800, 15 to 900, 15 to 1000, 15 to 1200, 15 to 1400, 15 to 1500, 15 to 1800, 15 to 2000, 15 to 2500, 15 to 3000, 50 to 100, 50 to 200, 50 to 300, 50 to 400, 50 to 500, 50 to 600, 50 to 700, 50 to 800, 50 to 900, 50 to 1000, 50 to 1200, 50 to 1400, 50 to 1500, 50 to 1800, 50 to 2000, 50 to 2500, 50 to 3000, 100 to 200, 100 to 300, 100 to 400, 100 to 500, 100 to 600, 100 to 700, 100 to 800, 100 to 900, 100 to 1000, 100 to 1200, 100 to 1400, 100 to 1500, 100 to 1800, 100 to 2000, 100 to 2500, 100 to 3000, 200 to 300, 200 to 400, 200 to 500, 200 to 600, 200 to 700, 200, to 800, 200 to 900, 200 to 1000, 200 to 1500, 200 to 3000, 500 to 1000, 500 to 1500, 500 to 2000, 500 to 2500, 500 to 3000, 1000 to 1500, 1000 to 2000, 1000 to 2500, 1000 to 3000, 1500 to 3000, 2500 to 3000, or 2000 to 3000 nucleotides).

In some embodiments, the poly-A tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the poly-A tail can be 10, 20, 30, 40, 50, 60, 70, 80, 90, or 100% greater in length than the nucleic acid or feature thereof. The poly-A tail can also be designed as a fraction of the nucleic acid to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of nucleic acids for Poly-A binding protein can enhance expression.

Additionally, multiple distinct nucleic acids can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr, and/or day 7 post-transfection.

In some embodiments, the nucleic acids of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant nucleic acid is assayed for stability, protein production, and other parameters including half-life at various time points. It has been discovered that the polyA-G quartet results in protein production from an mRNA equivalent to at least 75% of that seen using a poly-A tail of 120 nucleotides alone.

### Start Codon Region

The disclosure also includes a nucleic acid that comprises both a start codon region and the nucleic acid described herein (e.g., a nucleic acid comprising a nucleotide sequence encoding peptide epitopes). In some embodiments, the nucleic acids of the present disclosure can have regions that are analogous to or function like a start codon region.

In some embodiments, the translation of a nucleic acid can initiate on a codon that is not the start codon AUG. Translation of the nucleic acid can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety for this purpose).

As a non-limiting example, the translation of a nucleic acid begins on the alternative start codon ACG. As another non-limiting example, nucleic acid translation begins on the alternative start codon CTG or CUG. As yet another non-limiting example, the translation of a nucleic acid begins on the alternative start codon GTG or GUG.

Nucleotides flanking a codon that initiates translation such as, but not limited to, a start codon or an alternative start codon, are known to affect the translation efficiency, the length and/or the structure of the nucleic acid. (See, e.g., Matsuda and Mauro PLoS ONE, 2010 5:11; the contents of which are herein incorporated by reference in its entirety for this purpose). Masking any of the nucleotides flanking a codon that initiates translation can be used to alter the position of translation initiation, translation efficiency, length, and/or structure of a polynucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) nucleic acids and exon-junction complexes (EJCs) (See, e.g., Matsuda and Mauro describing masking agents LNA polynucleotides and EJCs (PLoS ONE, 2010 5:11); the contents of which are herein incorporated by reference in its entirety for this purpose).

In another embodiment, a masking agent can be used to mask a start codon of a nucleic acid in order to increase the likelihood that translation will initiate on an alternative start codon. In some embodiments, a masking agent can be used to mask a first start codon or alternative start codon in order to increase the chance that translation will initiate on a start codon or alternative start codon downstream to the masked start codon or alternative start codon.

In another embodiment, the start codon of a nucleic acid can be removed from the nucleic acid sequence in order to have the translation of the nucleic acid begin on a codon that is not the start codon. Translation of the nucleic acid can begin on the codon following the removed start codon or on a downstream start codon or an alternative start codon. In a non-limiting example, the start codon ATG or AUG is removed as the first 3 nucleotides of the nucleic acid sequence in order to have translation initiate on a downstream start codon or alternative start codon. The nucleic acid sequence where the start codon was removed can further comprise at least one masking agent for the downstream start codon and/or alternative start codons in order to control or attempt to control the initiation of translation, the length of the nucleic acid and/or the structure of the nucleic acid.

### Stop Codon Region

The disclosure also includes a nucleic acid that comprises both a stop codon region and the nucleic acid described herein (e.g., a nucleic acid encoding peptide epitopes). In some embodiments, the nucleic acids of the present disclosure can include at least two stop codons before the 3′ untranslated region (UTR). The stop codon can be selected from TGA, TAA and TAG in the case of DNA, or from UGA, UAA and UAG in the case of RNA. In some embodiments, the nucleic acids of the present disclosure include the stop codon TGA in the case or DNA, or the stop codon UGA in the case of RNA, and one additional stop codon. In a further embodiment the addition stop codon can be TAA or UAA. In another embodiment, the nucleic acids of the present disclosure include three consecutive stop codons, four stop codons, or more.

### Insertions and Substitutions

The disclosure also includes a nucleic acid of the present disclosure that further comprises insertions and/or substitutions.

In some embodiments, the 5′ UTR of the nucleic acid can be replaced by the insertion of at least one region and/or string of nucleosides of the same base. The region and/or string of nucleotides can include, but is not limited to, at least 3, at least 4, at least 5, at least 6, at least 7, or at least 8 nucleotides and the nucleotides can be natural and/or unnatural. As a non-limiting example, the group of nucleotides can include 5-8 adenine, cytosine, thymine, a string of any of the other nucleotides disclosed herein and/or combinations thereof.

In some embodiments, the 5′ UTR of the nucleic acid can be replaced by the insertion of at least two regions and/or strings of nucleotides of two different bases such as, but not limited to, adenine, cytosine, thymine, any of the other nucleotides disclosed herein, and/or combinations thereof. For example, the 5′ UTR can be replaced by inserting 5-8 adenine bases followed by the insertion of 5-8 cytosine bases. In another example, the 5′ UTR can be replaced by inserting 5-8 cytosine bases followed by the insertion of 5-8 adenine bases.

In some embodiments, the nucleic acid can include at least one substitution and/or insertion downstream of the transcription start site that can be recognized by an RNA polymerase. As a non-limiting example, at least one substitution and/or insertion can occur downstream of the transcription start site by substituting at least one nucleic acid in the region just downstream of the transcription start site (such as, but not limited to, +1 to +6). Changes to region of nucleotides just downstream of the transcription start site can affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety for this purpose). The modification, substitution, and/or insertion of at least one nucleoside can cause a silent mutation of the sequence or can cause a mutation in the amino acid sequence.

In some embodiments, the nucleic acid can include the substitution of at least 1, at least 2, at least 3, at least 4, at least 5, at least 6, at least 7, at least 8, at least 9, at least 10, at least 11, at least 12, or at least 13 guanine bases downstream of the transcription start site. In some embodiments, the nucleic acid can include the substitution of at least 1, at least 2, at least 3, at least 4, at least 5, or at least 6 guanine bases in the region just downstream of the transcription start site. As a non-limiting example, if the nucleotides in the region are GGGAGA, the guanine bases can be substituted by at least 1, at least 2, at least 3, or at least 4 adenine nucleotides. In another non-limiting example, if the nucleotides in the region are GGGAGA the guanine bases can be substituted by at least 1, at least 2, at least 3, or at least 4 cytosine bases. In another non-limiting example, if the nucleotides in the region are GGGAGA the guanine bases can be substituted by at least 1, at least 2, at least 3, or at least 4 thymine, and/or any of the nucleotides described herein.

In some embodiments, the nucleic acid can include at least one substitution and/or insertion upstream of the start codon. For the purpose of clarity, one of skill in the art would appreciate that the start codon is the first codon of the protein coding region whereas the transcription start site is the site where transcription begins. The nucleic acid can include, but is not limited to, at least 1, at least 2, at least 3, at least 4, at least 5, at least 6, at least 7, or at least 8 substitutions and/or insertions of nucleotide bases. The nucleotide bases can be inserted or substituted at 1, at least 1, at least 2, at least 3, at least 4, or at least 5 locations upstream of the start codon. The nucleotides inserted and/or substituted can be the same base (e.g., all A, or all C, or all T, or all G), two different bases (e.g., A and C, A and T, or C and T), three different bases (e.g., A, C and T, or A, C and T) or at least four different bases.

As a non-limiting example, the guanine base upstream of the coding region in the nucleic acid can be substituted with adenine, cytosine, thymine, or any of the nucleotides described herein. In another non-limiting example, the substitution of guanine bases in the nucleic acid can be designed so as to leave one guanine base in the region downstream of the transcription start site and before the start codon (see Esvelt et al. Nature (2011) 472(7344): 499-503; the contents of which is herein incorporated by reference in its entirety for this purpose). As a non-limiting example, at least 5 nucleotides can be inserted at 1 location downstream of the transcription start site but upstream of the start codon and the at least 5 nucleotides can be the same base type.

According to the present disclosure, two regions or parts of a chimeric nucleic acid may be joined or ligated, for example, using triphosphate chemistry. In some embodiments, a first region or part of 100 nucleotides or less is chemically synthesized with a 5′-monophosphate and terminal 3′-desOH or blocked OH. If the region is longer than 80 nucleotides, it may be synthesized as two or more strands that will subsequently be chemically linked by ligation. If the first region or part is synthesized as a non-positionally modified region or part using IVT, conversion to the 5′-monophosphate with subsequent capping of the 3′-terminus may follow. Monophosphate protecting groups may be selected from any of those known in the art. A second region or part of the chimeric nucleic acid may be synthesized using either chemical synthesis or IVT methods, e.g., as described herein. IVT methods may include use of an RNA polymerase that can utilize a primer with a modified cap. Alternatively, a cap may be chemically synthesized and coupled to the IVT region or part.

It is noted that for ligation methods, ligation with DNA T4 ligase followed by DNAse treatment (to eliminate the DNA splint required for DNA T4 Ligase activity) should readily prevent the undesirable formation of concatenation products.

The entire chimeric polynucleotide need not be manufactured with a phosphate-sugar backbone. If one of the regions or parts encodes a polypeptide, then it is preferable that such region or part comprise a phosphate-sugar backbone.

Ligation may be performed using any appropriate technique, such as enzymatic ligation, click chemistry, orthoclick chemistry, solulink, or other bioconjugate chemistries known to those in the art. In some embodiments, the ligation is directed by a complementary oligonucleotide splint. In some embodiments, the ligation is performed without a complementary oligonucleotide splint.

### Methods of Treatment

Provided herein are compositions (e.g., pharmaceutical compositions), methods, kits, and reagents for prevention and/or treatment of cancer in humans (e.g., subjects or patients) and other mammals. Nucleic acid cancer vaccines may be used as therapeutic or prophylactic agents in medicine to prevent and/or treat cancer. In exemplary aspects, the cancer vaccines of the present disclosure are used to provide prophylactic protection from cancer. Prophylactic protection from cancer can be achieved following administration of a cancer vaccine of the present disclosure. Vaccines can be administered once, twice, three times, four times, or more but it may be sufficient to administer the vaccine once (optionally followed by a single booster). It may also be desirable to administer the vaccine to an individual having cancer to achieve a therapeutic response. Dosing may need to be adjusted accordingly.

Once a cancer vaccine (e.g., a nucleic acid cancer vaccine) is synthesized, it is administered to the patient. In some embodiments the vaccine is administered on a schedule for up to two months, up to three months, up to four month, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments the schedule is weekly for the first 3 weeks and then monthly thereafter. The schedule may be determined or varied by one of skill in the art (e.g., a medical doctor) depending on the individual patient or subject's criteria (e.g., weight, age, type of cancer, etc.).

The vaccine may be administered by any route. In some embodiments the vaccine is administered by an intradermal, intramuscular, intravascular, intratumoral, and/or subcutaneous route.

In some embodiments, the nucleic acid cancer vaccine may also be administered with an anti-cancer therapeutic agent. The nucleic acid cancer vaccine and other therapeutic agent may be administered simultaneously or sequentially. When the other therapeutic agents are administered simultaneously they can be administered in the same or separate formulations, but are administered at the same time. The other therapeutic agents are administered sequentially with one another and with the nucleic acid cancer vaccine, when the administration of the other therapeutic agents and the nucleic acid cancer vaccine is temporally separated. The separation in time between administrations of these compounds may be a matter of minutes or it may be longer, e.g., hours, days, weeks, months. Other therapeutic agents include but are not limited to anti-cancer therapeutic, adjuvants, cytokines, antibodies, antigens, etc.

At any point in the treatment the patient may be examined to determine whether the mutations in the vaccine are still appropriate. Based on that analysis the vaccine may be adjusted or reconfigured to include one or more different mutations or to remove one or more mutations.

In exemplary embodiments, a cancer vaccine containing RNA polynucleotides as described herein can be administered to a subject (e.g., a mammalian subject, such as a human subject), and the RNA polynucleotides are translated in vivo to produce an antigenic polypeptide.

The cancer vaccines may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a composition containing a cancer vaccine that contains a polynucleotide that has at least one a translatable region encoding an antigenic polypeptide.

An “effective amount” of a cancer RNA vaccine may be provided based, at least in part, on the target tissue, target cell type, means of administration, physical characteristics of the polynucleotide (e.g., size, and extent of modified nucleosides) and other components of the cancer vaccine, and other determinants. In general, an effective amount of the cancer vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the cancer vaccine), increased protein translation from the polynucleotide, decreased nucleic acid degradation (as demonstrated, for example, by increased duration of protein translation from a modified polynucleotide), or altered antigen specific immune response of the host cell.

Cancer vaccines may be administered prophylactically or therapeutically as part of an active immunization scheme to healthy individuals or early in cancer or during active cancer after onset of symptoms. In some embodiments, the amount of RNA vaccines of the present disclosure provided to a cell, a tissue or a subject may be an amount effective for immune prophylaxis.

Cancer vaccines may be administered with other prophylactic or therapeutic compounds in addition to checkpoint inhibitors. As a non-limiting example, a prophylactic or therapeutic compound may be an immune potentiator or a booster. As used herein, when referring to a composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

The cancer vaccines may be utilized in various settings depending on the severity of the cancer or the degree or level of unmet medical need. As a non-limiting example, the cancer vaccines may be utilized to treat any stage of cancer.

In some embodiments the cancer vaccines and/or checkpoint inhibitors may be used to treat PD-L1 positive tumors. In other embodiments the cancer vaccines and/or checkpoint inhibitors may be used to treat PD-L1 negative tumors. While emerging data support the use of PD-1 inhibitors such as pembrolizumab in tumors where PD-L1 expression can be demonstrated, the use of the combinations of the invention in treating PD-1 “negative” tumors is envisioned. Mechanistically, there is an adaptive component to PD-L1 expression by tumors, i.e., tumors may initially appear PD-L1 negative but upregulate PD-L1 expression in response to IFN-γ secretion by infiltrating tumor lymphocytes. This has translated clinically, such that the response rates of PD-L1 negative tumors to the combination of PD-1 and CTLA-4 blockade is higher than the response rate to single agent PD-1 inhibitors in both cutaneous melanoma and lung cancer. Aspects of the invention relate to the use of a personalized cancer vaccine to induce PD-L1 expression in PD-L1 low tumors, in combination with a PD-1 inhibitor.

In some embodiments that the cancer vaccines and/or checkpoint inhibitors may be used to treat tumors having a high tumor mutation burden. Thus in some embodiments a pool of subjects may be tested for TMB and the subjects having a TMB value over a threshold level may be treated with the combination therapy of the invention.

A non-limiting list of cancers that the cancer vaccines may treat is presented below. Peptide epitopes or antigens may be derived from any antigen of these cancers or tumors. Such epitopes may be referred to as cancer or tumor antigens. Cancer cells may differentially express cell surface molecules during different phases of tumor progression. For example, a cancer cell may express a cell surface antigen in a benign state, yet down-regulate that particular cell surface antigen upon metastasis. As such, it is envisioned that the tumor or cancer antigen may encompass antigens produced during any stage of cancer progression. The methods of the disclosure may be adjusted to accommodate for these changes. For instance, several different cancer vaccines may be generated for a particular patient. For instance, a first vaccine may be used at the start of the treatment. At a later time point, a new cancer vaccine may be generated and administered to the patient to account for different antigens being expressed.

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present disclosure include, but are not limited to, those listed below (for a review of such disorders, see Fishman et al., 1985, Medicine, 2d Ed., J.B. Lippincott Co., Philadelphia). Cancers for use with the instantly described methods and compositions may include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas; tumor mutational burden high tumors; choriocarcinomas; stromal tumors and germ cell tumors; thyroid cancer including thyroid adenocarcinoma and medullar carcinoma; and renal cancer including adenocarcinoma and Wilms' tumor. In some embodiments that cancer is any one of melanoma, bladder carcinoma, HPV negative HNSCC, NSCLC, SCLC, MSI-High tumors, or TMB (tumor mutational burden) High cancers.

In some embodiments, the cancer is selected from the group consisting of non-small cell lung cancer (NSCLC), small cell lung cancer, melanoma, bladder urothelial carcinoma, HPV-negative head and neck squamous cell carcinoma (HNSCC), and a solid malignancy that is microsatellite high (MSI H)/mismatch repair (MMR) deficient. In some embodiments, the NSCLC lacks an EGFR sensitizing mutation and/or an ALK translocation. In some embodiments, the solid malignancy that is microsatellite high (MSI H)/mismatch repair (MMR) deficient is selected from the group consisting of colorectal cancer, stomach adenocarcinoma, esophageal adenocarcinoma, and endometrial cancer.

Pembrolizumab monotherapy (10 mg/kg dosed every 2 weeks) has been used in subjects with advanced solid tumors that express PD-L1 which have not responded to current therapy or for which current therapy is not appropriate. For instance, in subjects with small cell lung cancer (SCLC) it was concluded that pembrolizumab is generally well tolerated and has promising antitumor activity in subjects with PD-L1+ SCLC who have progressed on prior platinum-based therapy. In another study of patients with advanced urothelial cancer who were given 10 mg/kg pembrolizumab every 2 weeks, it was concluded that pembrolizumab demonstrates durable antitumor activity in subjects with advanced urothelial cancer. The combination therapy is also useful for treating Microsatellite Instability High Cancers, such as colorectal cancer, endometrial tumors, adenocarcinoma of the stomach or gastro-esophageal junction or gastric cancer.

Provided herein are pharmaceutical compositions including cancer vaccines and RNA vaccine compositions and/or complexes optionally in combination with one or more pharmaceutically acceptable excipients. Cancer vaccines may be formulated or administered alone or in conjunction with one or more other components as described herein.

In other embodiments the cancer vaccines described herein may be combined with any other therapy useful for treating the patient. For instance a patient may be treated with the cancer vaccine and an anti-cancer agent. Thus, in one embodiment, the methods of the disclosure can be used in conjunction with one or more cancer therapeutics, for example, in conjunction with an anti-cancer agent, a traditional cancer vaccine, chemotherapy, radiotherapy, etc. (e.g., simultaneously, or as part of an overall treatment procedure). Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previous treatment methods. Any agent or therapy (e.g., traditional cancer vaccines, chemotherapies, radiation therapies, surgery, hormonal therapies, and/or biological therapies/immunotherapies) which is known to be useful, or which has been used or is currently being used for the prevention or treatment of cancer can be used in combination with a composition of the disclosure in accordance with the disclosure described herein. One of ordinary skill in the medical arts can determine an appropriate treatment for a subject.

Examples of such agents (i.e., anti-cancer agents) include, but are not limited to, DNA-interactive agents including, but not limited to, the alkylating agents (e.g., nitrogen mustards, e.g., Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin Interactive agents including, but not limited to, colcbicine, Vincristine and Vinblastine, both alkaloids and Paclitaxel and cytoxan; hormonal agents including, but not limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1.alpha., IL-1 β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-.γ, and Uteroglobins (U.S. Pat. No. 5,696,092); anti-angiogenics including, but not limited to, agents that inhibit VEGF (e.g., other neutralizing antibodies), soluble receptor constructs, tyrosine kinase inhibitors, antisense strategies, RNA aptamers and ribozymes against VEGF or VEGF receptors, immunotoxins and coaguligands, tumor vaccines, and antibodies.

Specific examples of anti-cancer agents which can be used in accordance with the methods of the disclosure include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interieukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs which may be used with the instant compositions and methods include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RH retinamide; rubiginone B 1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The disclosure also encompasses administration of a composition comprising a cancer vaccine in combination with radiation therapy comprising the use of x-rays, gamma rays and other sources of radiation to destroy the cancer cells. In certain embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer (e.g., by a physician). For instance, a patient with ovarian cancer may be administered a prophylactically or therapeutically effective amount of a composition comprising a cancer vaccine in combination with a prophylactically or therapeutically effective amount of one or more other agents useful for ovarian cancer therapy, including but not limited to, intraperitoneal radiation therapy, such as P32 therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Cancer therapies and their dosages, routes of administration and recommended usage are known in the art and have been described in such literature as the Physician's Desk Reference (56th ed., 2002).

In some embodiments the cancer therapeutic agent is a targeted therapy. The targeted therapy may be a BRAF inhibitor such as vemurafenib (PLX4032) or dabrafenib. The BRAF inhibitor may be PLX 4032, PLX 4720, PLX 4734, GDC-0879, PLX 4032, PLX-4720, PLX 4734 and Sorafenib Tosylate. BRAF is a human gene that makes a protein called B-Raf, also referred to as proto-oncogene B-Raf and v-Raf murine sarcoma viral oncogene homolog B 1. The B-Raf protein is involved in sending signals inside cells, which are involved in directing cell growth. Vemurafenib, a BRAF inhibitor, was approved by FDA for treatment of late-stage melanoma.

In other embodiments the cancer therapeutic agent is a cytokine. In yet other embodiments the cancer therapeutic agent is a vaccine comprising a population based tumor specific antigen. In yet other embodiments, the cancer therapeutic agent is vaccine containing one or more traditional antigens expressed by cancer-germline genes (antigens common to tumors found in multiple patients, also referred to as “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

The p53 gene (official symbol TP53) is mutated more frequently than any other gene in human cancers. Large cohort studies have shown that, for most p53 mutations, the genomic position is unique to one or only a few patients and the mutation cannot be used as recurrent neoantigens for therapeutic vaccines designed for a specific population of patients. A small subset of p53 loci do, however, exhibit a “hotspot” pattern (described elsewhere herein), in which several positions in the gene are mutated with relatively high frequency. Strikingly, a large portion of these recurrently mutated regions occur near exon-intron boundaries, disrupting the canonical nucleotide sequence motifs recognized by the mRNA splicing machinery.

Mutation of a splicing motif can alter the final mRNA sequence even if no change to the local amino acid sequence is predicted (i.e., for synonymous or intronic mutations). Therefore, these mutations are often annotated as “noncoding” by common annotation tools and neglected for further analysis, even though they may alter mRNA splicing in unpredictable ways and exert severe functional impact on the translated protein. If an alternatively spliced isoform produces an in-frame sequence change (i.e., no pretermination codon (PTC) is produced), it can escape depletion by nonsense-mediated mRNA decay (NMD) and be readily expressed, processed, and presented on the cell surface by the HLA system. Further, mutation-derived alternative splicing is usually “cryptic”, i.e., not expressed in normal tissues, and therefore may be recognized by T-cells as non-self neoantigens.

In some instances, the cancer therapeutic agent is a vaccine which includes one or more neoantigens which are recurrent polymorphisms (“hot spot mutations”). For example, among other things, the present disclosure provides neoantigen peptide sequences resulting from certain recurrent somatic cancer mutations in p53.

### Formulations

Cancer vaccines (e.g., nucleic acid cancer vaccines such as mRNA cancer vaccines) may be formulated or administered in combination with one or more pharmaceutically-acceptable excipients. As a non-limiting set of examples, cancer vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with cancer vaccines (e.g., for transplantation into a subject), hyaluronidase, nanoparticle mimics and combinations thereof.

In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety for this purpose).

In some embodiments, cancer vaccines are administered to humans, human patients or subjects. For the purposes of the present disclosure, the phrase “active ingredient” generally refers to the cancer vaccines or the nucleic acids contained therein, for example, RNA (e.g., mRNA) encoding antigenic polypeptides.

Formulations of the vaccine compositions described herein may be prepared by any method known or hereafter developed in the art of pharmacology. In general, such preparatory methods include the step of bringing the active ingredient (e.g., nucleic acids such as mRNA) into association with an excipient and/or one or more other accessory ingredients, and then, if necessary and/or desirable, dividing, shaping and/or packaging the product into a desired single- or multi-dose unit.

The formulation of any of the compositions disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

In some embodiments, the compositions disclosed herein may be formulated as lipid nanoparticles (LNP). Accordingly, the present disclosure also provides nanoparticle compositions comprising (i) a lipid composition comprising a delivery agent, and (ii) a nucleic acid encoding one or more peptide epitopes. In such nanoparticle composition, the lipid composition disclosed herein can encapsulate the nucleic acid encoding one or more peptide epitopes.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid.

The ratio between the lipid composition and the cancer vaccine may be from about 10:1 to about 60:1 (wt/wt). In some embodiments, the ratio between the lipid composition and the nucleic acid may be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the cancer vaccine is about 20:1 or about 15:1.

In one embodiment, the cancer vaccine (e.g., the nucleic acid cancer vaccine) may be comprised in lipid nanoparticles such that the lipid:polynucleotide weight ratio is 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the cancer vaccine (e.g., the nucleic acid cancer vaccine) may be comprised in lipid nanoparticles in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos.: WO2017075531, WO2015199952, WO2013086354, or WO2013116126, or selected from formulae CLI-CLXXXXII of U.S. Pat. No. 7,404,969; each of which is hereby incorporated by reference in its entirety for this purpose.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure. In addition to these, an ionizable lipid may also be a lipid including a cyclic amine group.

Vaccines of the present disclosure are typically formulated into lipid nanoparticles. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid. For example, the lipid nanoparticle may comprise a molar ratio of 20-50%, 20-40%, 20-30%, 30-60%, 30-50%, 30-40%, 40-60%, 40-50%, or 50-60% ionizable amino lipid. In some embodiments, the lipid nanoparticle comprises a molar ratio of 20%, 30%, 40%, 50, or 60% ionizable amino lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable amino lipid, 5-25% non-cationic lipid, 25-55% sterol, and 0.5-15% PEG-modified lipid.

In some embodiments, an ionizable amino lipid of the disclosure comprises a compound of Formula (I):

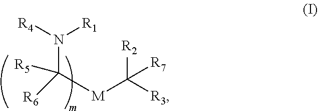

or a salt or isomer thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C1-3 alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and

H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, a subset of compounds of Formula (I) includes those of Formula (IA):

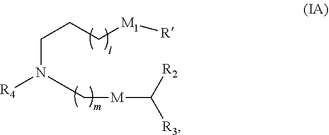

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

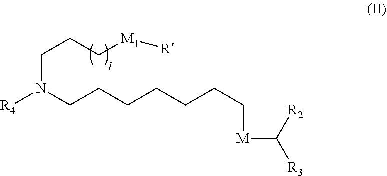

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

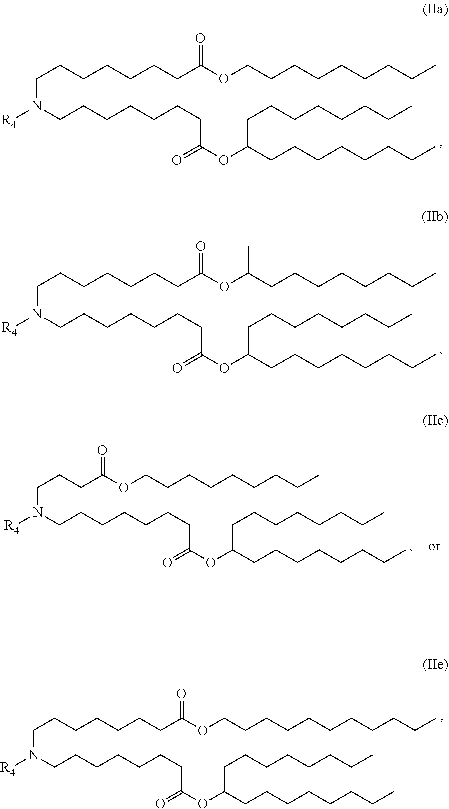

or a salt or isomer thereof, wherein R₄ is as described herein.

In some embodiments, a subset of compounds of Formula (I) includes those of Formula (IId):

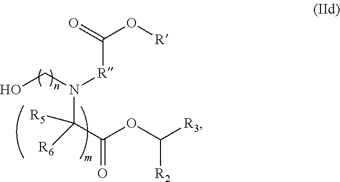

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

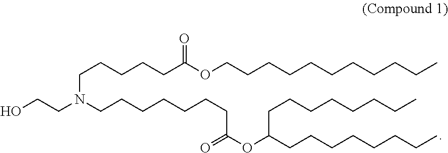 [SMILES: CCCCCCCCCCCOC(=O)CCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

In one embodiment, the lipid may be a cleavable lipid such as those described in International Publication No. WO2012170889, herein incorporated by reference in its entirety for this purpose. In one embodiment, the lipid may be synthesized by methods known in the art and/or as described in International Publication Nos. WO2013086354; the contents of which is herein incorporated by reference in its entirety for this purpose.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle composition.

Relative amounts of the active ingredient (e.g., the nucleic acid cancer vaccine), the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the package containing the pharmaceutical product contains 0.1 mg to 1 mg of nucleic acid (e.g., mRNA). In some embodiments, the package containing the pharmaceutical product contains 0.35 mg of nucleic acid (e.g., mRNA). In some embodiments, the concentration of the nucleic acid (e.g., mRNA) is 1 mg/mL.

In some embodiments, the nucleic acid (e.g., mRNA) vaccine compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013078199, herein incorporated by reference in its entirety). In some embodiments, the nucleic acid (e.g., mRNA) vaccine is administered at a dosage level sufficient to deliver 0.0100 mg, 0.025 mg, 0.040 mg, 0.050 mg, 0.075 mg, 0.100 mg, 0.125 mg, 0.130 mg, 0.150 mg, 0.175 mg, 0.200 mg, 0.225 mg, 0.250 mg, 0.275 mg, 0.300 mg, 0.325 mg, 0.350 mg, 0.375 mg, 0.390 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. In some embodiments, the nucleic acid (e.g., mRNA) vaccine is administered at a dosage level sufficient to deliver between 10 μg and 400 μg of the mRNA vaccine to the subject. In some embodiments, the nucleic acid (e.g., mRNA) vaccine is administered at a dosage level sufficient to deliver at least 0.033 mg, at least 0.040 mg, at least 0.1 mg, at least 0.13 mg, at least 0.2 mg, at least 0.39 mg, at least 0.4 mg, or at least 1.0 mg to the subject.

In some embodiments, the nucleic acid (e.g., mRNA) vaccine is administered at a dosage level sufficient to deliver at least 1.0 mg, at least 1.2 mg, at least 1.4 mg, at least 1.6 mg, at least 1.8 mg, or at least 2.0 mg, at least to the subject. In some embodiments, the nucleic acid (e.g., mRNA) vaccine is administered at a dosage level sufficient to deliver at least 2.0 mg, at least 2.2 mg, at least 2.4 mg, at least 2.6 mg, at least 2.8 mg, or at least 3.0 mg, at least to the subject. In some embodiments, the nucleic acid (e.g., mRNA) vaccine is administered at a dosage level sufficient to deliver at least 3.0 mg, at least 3.2 mg, at least 3.4 mg, at least 3.6 mg, at least 3.8 mg, or at least 4.0 mg, at least to the subject. In some embodiments, the nucleic acid (e.g., mRNA) vaccine is administered at a dosage level sufficient to deliver at least 4.0 mg, at least 4.2 mg, at least 4.4 mg, at least 4.6 mg, at least 42.8 mg, or at least 5.0 mg, at least to the subject.

The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, the nucleic acid (e.g., mRNA) vaccine compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the nucleic acid (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the nucleic acid (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.040 mg, 0.050 mg, 0.075 mg, 0.100 mg, 0.125 mg, 0.130 mg, 0.150 mg, 0.175 mg, 0.200 mg, 0.225 mg, 0.250 mg, 0.275 mg, 0.300 mg, 0.325 mg, 0.350 mg, 0.375 mg, 0.390 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a nucleic acid (e.g., mRNA) vaccine composition may be administered three or four times, or more. In some embodiments, the mRNA vaccine composition is administered once a day every three weeks.

In some embodiments, the nucleic acid (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments the nucleic acid (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject.

The methods and compositions described herein are not limited in its application to the details of construction and the arrangement of components set forth in the following description. The methods and compositions described herein are capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1: In Vivo Study to Evaluate the Safety and Immunogenicity of the mRNA Vaccine

Personalized cancer vaccines were created using the unique mutational profile of an individual patient's tumor matched to their specific HLA-type. Each unique mRNA vaccine comprised a single strand of mRNA encoding up to 20 neoantigens and formulated in lipid nanoparticles (LNPs) for intramuscular (IM) injection. Such mRNA vaccines are thought to prime, activate, and expand a diverse population of neoantigen-specific T cells for the treatment of solid tumors. The safety and tolerability of the mRNA vaccine was assessed both as monotherapy and in combination with pembrolizumab.

### Repeat Dose Toxicology Study (Sprague Dawley Rats)

The mRNA cancer vaccine formulation has been evaluated in a GLP repeat dose toxicology study in Sprague Dawley rats. The subjects were administered five doses total. Specifically, the mRNA vaccine was administered by a weekly IM injection at 12, 35, and 117 μg per dose for a total of 5 doses, followed by a 2-week post-study recovery period. The mRNA vaccine was tolerated at all dose levels (no mRNA vaccine-related mortality was observed). In addition, no changes in serum chemistry, urinalysis, or ocular findings were noted. Weekly administration of the mRNA vaccine to Sprague Dawley rats (both male and female) at dose levels of 12, 35, and 117 μg was associated with dose-dependent slight to moderate decreases in body weight gain and associated food consumption at doses greater than or equal to 12 μg. Clinical observations of injection site reactions were observed at all doses and included erythema (slight to mild) and edema (slight to moderate) that increased in incidence and severity with increasing dose. These clinical observations were associated with gross and microscopic changes at the injection site and with enlargement of the lymph nodes. Hematology changes consistent with inflammation were also observed and included mild to moderate increases in the WBC count (driven primarily by increased neutrophils) and decreases in lymphocytes. Systemic effects were primarily considered responses to local inflammation at the injection site. All changes were fully or partially resolved (absolute body weights remained lower than controls, mononuclear cell infiltration and chronic inflammation at the injection site, lymphoid hyperplasia, and increased hematopoiesis in the spleen and bone marrow) following the 2-week recovery period.

Based on these findings the No Observed Adverse Effect Level (NOAEL) of 0.035 mg for the IM route of administration in male and female rats represents an approximate 24-fold safety margin over the proposed human starting dose of 0.04 mg, and an approximate 2-fold safety margin over the proposed human testing dose of 0.39 mg.

In vivo Human Analysis

A monotherapy arm of the Phase 1 study assessed the safety, tolerability and immunogenicity of mRNA vaccine monotherapy in adjuvant (disease-free, post-resection) cancer patients. A subject in the adjuvant setting may derive benefit, as the mRNA vaccine may help to prevent the patient's cancer from recurring by teaching their immune system to better identify cancerous tissue derived from the original lesion.

The combination arm of the Phase 1 study assessed the safety, tolerability and immunogenicity of the mRNA vaccine in combination with pembrolizumab in cancer patients with unresectable (locally advanced or metastatic) solid tumors. Pembrolizumab is approved for use in several cancer types, and is under investigation in several phases of clinical development for many more. Despite much progress in the field of immune oncology therapeutics, an unmet medical need exists, as not all subjects respond to pembrolizumab therapy, most responses are not complete, and it is only approved for use in limited tumor types. Therefore, the mRNA vaccine may have increased efficacy in subjects with a variety of advanced unresectable solid malignancies. Subjects in the combination arm will have unresectable (locally advanced or metastatic) disease that is in urgent need of treatment. Therefore, as soon as patients have passed screening and are eligible for the study, they will start pembrolizumab dosing while their unique mRNA vaccines are being manufactured. As soon as the vaccine is ready, subjects will start combination treatment. This design ensures that treatment is initiated for patients with unresectable tumors in a timely fashion.

A further combination arm of the Phase I study assessed the safety, tolerability and immunogenicity of the mRNA vaccine in combination with pembrolizumab in adjuvant (disease-free, post-resection) melanoma patients. A subject in the adjuvant setting may derive benefit, as the mRNA vaccine may help to prevent the patient's cancer from recurring by teaching their immune system to better identify cancerous tissue derived from the original lesion.

Risk to subjects receiving the mRNA vaccine in the monotherapy arm or the combination arm of the study is expected to be low and primarily involving mild to moderate injection site reactions (ISRs) which have been observed in animal studies and generally observed and expected for other IM administered vaccines. These local reactions may consist of transient and dose-dependent pain, swelling, and erythema. Possible mild to moderate systemic reactions, which are also transient, may include fever, fatigue, chills, headache, myalgias, and arthralgias. Such adverse events (AEs) may, in part, be due to the poor biodegradability of LNP formulations used in other vaccine studies. The LNP that encapsulates the mRNA vaccine is designed to have improved biodegradability and thus may have improved tolerability. In addition, other AEs that have been generally associated with approved IM administered vaccines have included mild hematological and clinical chemistry abnormalities, which are usually reversible.

### Clinical Data—Monotherapy Arm of Phase 1 Trial

Thirteen (13) patients have been treated with monotherapy (administration of the personalized mRNA cancer vaccine) using the following dosages: 0.04 mg (3 patients), 0.13 mg (6 patients), 0.39 mg (3 patients), and 1.0 mg (1 patient). Of the 13 patients, 12 of the 13 are recurrence-free, either in follow-up or still on treatment. A summary of the injections and dosages is provided in Table 1 below. There have been no dose-limiting toxicities or significant related toxicities observed in the patients. mRNA cancer vaccine was administered for a maximum of nine 21-day cycles (with dosing on C1D1, C2D1, C3D1, and C4D1; with C # referring to dose number of the cancer vaccine, P # referring to the dose number of the pembrolizumab and D # referring to the day of the 21 day cycle).

TABLE 1
Dose levels of mRNA cancer vaccine

| | |
|---|---|
| mRNA cancer vaccine | mRNA cancer vaccine |
| dose level^{a} | injection volume |

| | | |
|---|---|---|
| 0.04 | mg | One injection of 0.3 mL |
| 0.13 | mg | One injection of 0.3 mL |
| 0.39 | mg | One injection of 0.3 mL |
| 1.00 | mg^{b} | Two injections of 0.3-0.5 mL^{c} |

^{b}1.00 mg is an example dose level, different dose level(s) above 0.39 mg may be used, up to a maximum of 1.17 mg mRNA cancer vaccine.
^{c}The exact dose volume for any dose level above 0.39 mg may be up to two injections of between 0.3 mL to 0.5 mL.

### RNA-Seq Analysis and Biomarker Analysis:

An RNA-seq analysis of three patients at the 0.39 mg dose level was undertaken to explore tumor mutational burden (TMB), expression levels of selected checkpoint molecules, and gene expression profiles (GEPs).

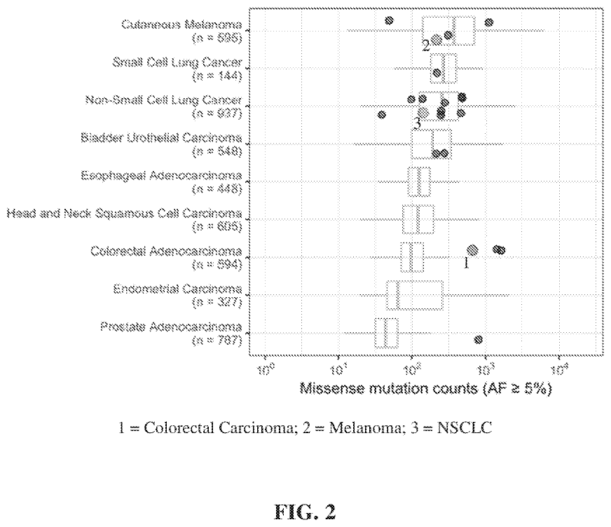

Tumor mutational burden (TMB) was determined for the three patients in the context of average TMB across approximately 15,000 human cancers (Chang et al., Cancer Discovery, 2017) and previously analyzed patient data (FIG. 2). For a patient with melanoma and a patient with non-small cell lung carcinoma (NSCLC), the TMB values were found to fall within the typical range (1.5*IQR (interquartile range)) of TMB reported in corresponding tumor types. The tumor from a patient with colorectal adenocarcinoma was found to be microsatellite instability (MSI)-high, and accordingly had a TMB above the typical range reported in that tumor type. Table 2, below, shows the number of non-synonymous mutations of the three patients. Of note, several studies have linked TMB^{hi} tumors with clinical response to checkpoint inhibitors (CPIs); most recently a comprehensive analysis by Cristescu et al. found that combining gene expression profiling (GEP) score or PD-L1 levels with TMB may allow for identification of patients who will respond to pembrolizumab (Cristescu et al. Science, 2018).

TMB, PD-L1 gene expression level, and T cell-inflamed gene expression profile (GEP) were analyzed for pre-treatment biopsies of the patients (over 40 patients) in conjunction with vaccine monotherapy or in combination with pembrolizumab using Whole Exome Sequencing (WES) and RNA-seq data. Antigen specific T cells responses were evaluated in patient PBMCs.

Compared to a pan-cancer cohort with ˜15,000 tumors [Chang M T, Bhattarai T S, Schram A M, et al. Accelerating Discovery of Functional Mutant Alleles in Cancer. Cancer Discov. 2018; 8(2):174-183], the TMB of most patients was within the tumor type-specific range (within 1.5× interquartile above the Q3 and below Q1), while MSI-high patients of colon, prostate and squamous cell cancers are outliers, with significantly higher TMB.

TABLE 2
Tumor Mutation Burden of Three 0.39 mg Dose Patients

| | No. of Non-Synonymous |
|---|---|
| Tumor Type | Mutations |
| MSI^{hi} Colorectal | 662 |
| Carcinoma (CRC) | |
| Melanoma | 215 |
| Non-Small Cell Lung | 142 |
| Carcinoma (NSCLC) | |

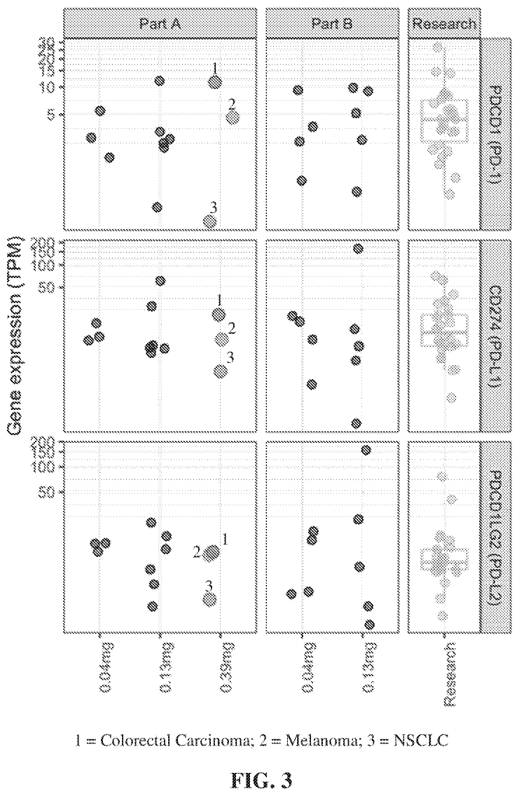

The expression levels of PD-1, PD-L1, and PD-L2 were examined relative to test samples and previously reported patient samples (FIG. 3). The test samples used were 26 tumors purchased from a biobank spanning 13 tumor types. For a patient with CRC and a patient with NSCLC, the expression levels of genes of interest were within the range observed in the test samples. However, an exception was observed in a patient having melanoma. That patient had much lower levels of PD-1 than were measured in test samples and previously analyzed patient samples, as well as relatively lower levels of PD-L1 and PD-L2. The mRNA level of PD-1 has previously been reported to positively correlate to overall response rates to CPIs (Pare, L. et al. Ann. Oncol, 2018), and the protein level of PD-L1 is also a predictive biomarker for response to CPIs (Patel and Kurzrock, Mol. Cancer Ther. 2015). Though several studies report a high correlation between gene expression of PD-L1 and protein levels of PD-L1 (Schultheis et al, European Journal of Cancer, 2015; Jaeger S, et al. AACR 106th Annual Meeting, 2015; Paluch B E, et al. Oncotarget, 2017), gene expression of PD-L1 is not currently an approved predictive biomarker for response to CPIs. Overall, the expression level of PD-1, PD-L1 and PD-L2 were extracted from RNA-seq. The patients with high PD-L1 expression level, also tend to have high level of PD-L2 (r=0.78, p<6.2e-15, Pearson correlation). The expression level of PD-L1 and PD-1 are also correlated, but with a lower correlation (r=0.57, p<4.1e-7).

The gene expression profile (GEP)_of three 0.39 mg dose patients was examined (data not shown). The T cell-inflamed gene expression profile (GEP) was developed as a pan-tumor predictive biomarker for predicting response to pembrolizumab (Ayers et al., J. Clin. Invest, 2017). Recently, it has been reported that GEP can stratify human cancers into groups with different clinical responses to pembrolizumab monotherapy (Cristescu et al. Science, 2018). The GEP score reported in these publications was developed using the Nanostring platform looking at 18 specific genes. Seventeen of the 18 genes reported by Ayers et al. were used for the analysis. The analysis system used requires RNA-seq data, rather than the Nanostring platform and HLA-DRB1 is not annotated in that system. Three patients in the 0.39 mg dose cohort were found to have a wide range of levels of T cell-inflamed gene expression; a patient with CRC was found to have the highest GEP level evaluated in this cohort, and a patient with melanoma was found to have the lowest GEP levels across all patient and test samples analyzed to date.

The cytolytic activity score (CYT) were introduced to quantify local immune infiltrate [Rooney M S, Shukla S A, Wu C J, Getz G, Hacohen N. Molecular and genetic properties of tumors associated with local immune cytolytic activity. Cell. 2015; 160(1-2):48-61.], which showed good correlation to GEP score (r=0.84, p<2.2e-16) in these patients. Patients were stratified by TMB (<100 mutations) and GEP score (below the top tertile of data). ˜30% of the analyzed patients are in GEP^{high} and TMB^{high} group.

Thus, next-generation sequencing was used to characterize mutation landscape, expression level of key genes, and tumor microenvironment of pre-treatment biopsies. By correlating to neoantigen-specific T cell responses post-vaccination, this data provides important biomarkers for personalized neoantigen cancer vaccines.

### Antigen-Specific T Cell Response Analysis

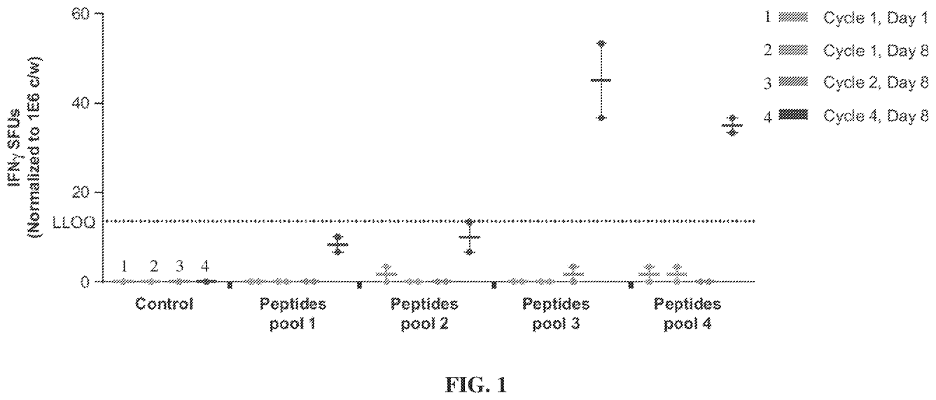

As shown in FIG. 1, for a patient with melanoma at the 0.13 mg dose, potential antigen-specific T cell responses have been detected after the fourth dose of the mRNA vaccine. This was measured by restimulating unexpanded peripheral blood mononuclear cells with sets of peptides corresponding to neoantigens encoded by the patient-specific mRNA vaccine. In FIG. 1, individual data points indicate technical replicates.

Among the 10 patients for which cycle 4 PBMC data has been obtained to date, in the monotherapy arm of the trial, the three patients at the 0.04 mg dose did not yet exhibit detectable immunogenicity. In contrast, at the 0.13 mg dose 5 of the 6 patients had detectable immunogenicity (above LOD by C₄) and 1 had immunogenicity greater than LLOQ. One patient had responses above LOD and LLOQ at baseline, as well as response above LOD at C4D8 for the sample peptide pool. Another patient had responses above LOD at C2D8 and C4D8 for the same peptide pool. A third patient had a response of greater than LOD at baseline for one peptide pool, and at C4D8 for a different peptide pool.

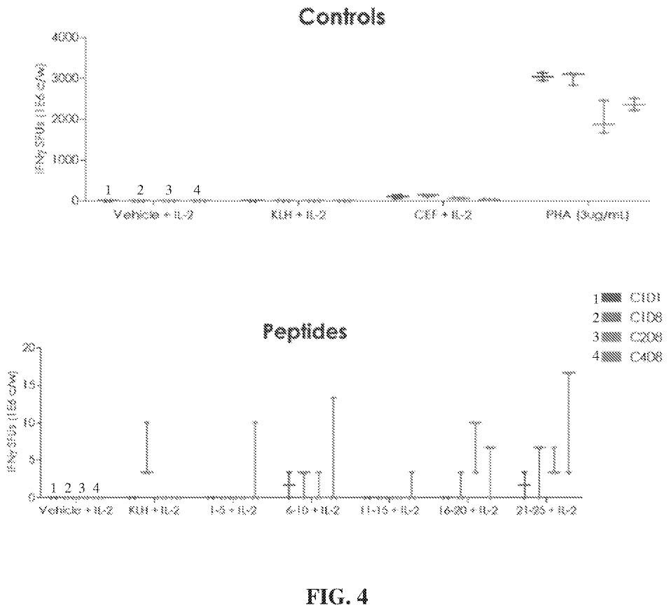

Data from three patients at the 0.39 mg dose level was obtained. As shown in FIG. 4, for a patient with CRC at a 0.39 mg dose level, antigen-specific T cell responses above the assays' lower limit of quantification (LLOQ) (13.5 SFU/1e10⁶) were not detected. The time points assessed were baseline (C1D1), 7 days (7d) post first dose (C1D8), 7d post second dose (C2D8), and 7d post fourth dose (C4D8). Table 3, below, summarizes the average spot forming unit (SFU) values for each peptide pool at each collection point for the patient. Note that there was a protocol dosing violation for this subject on C3D1. All other doses of the vaccine were correctly administered.

TABLE 3
Average Spot-Forming Unit Values

| | C1D1 | C1D8 | C2D8 | C4D8 |
|---|---|---|---|---|
| Vehicle | 0.0 | 0.0 | 0.0 | 0.0 |
| KLH | 0.0 | 5.6 | 0.0 | 0.0 |
| Peptides 1-5 | 0.0 | 0.0 | 0.0 | 3.3 |
| Peptides 6-10 | 1.7 | 2.2 | 1.1 | 4.4 |
| Peptides 11-15 | 0.0 | 0.0 | 0.0 | 1.1 |
| Peptides 16-20 | 0.0 | 1.1 | 7.8 | 4.4 |
| Peptides 21-25 | 1.7 | 4.4 | 4.4 | 12.2 |

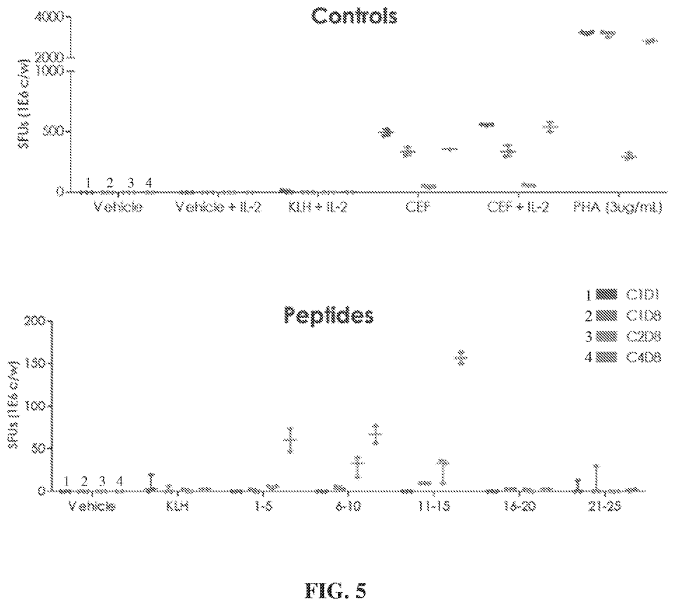

As shown in FIG. 5, for a patient with melanoma at the 0.39 mg dose level, antigen-specific T cell responses above the assay LLOQ (13.5 SFU/1e10⁶) ranging from 26.7-156.7 SFU/1e10⁶ cells/well were detected post ex vivo restimulation of PBMCs with peptides corresponding to neoantigens encoded in the vaccine at C2D8 and C4D8. A response of 60 SFU/1e10⁶ cells/well was detected against peptide pool 1-5 at C4D8. A response of 30 SFU/1e10⁶ cells/well was detected against peptide pool 6-10 at C2D8 which increased to 66.7 SFU/1e10⁶ cells/well at C4D8. Similarly, the responses against peptide pool 11-15 grew from 26.7 SFU/1e10⁶ cells/well at C2D8 to 156.7 SFU/1e10⁶ cells/well by C4D8. The observed responses were approximately 2-3 times greater in magnitude after the fourth dose. Antigen-specific responses above LLOQ were not detected against these peptide pools at earlier time points, nor for peptide pool 16-20 and 21-25 at any time point. Table 4 summarizes average spot forming unit (SFU) values for each peptide pool at each collection point for this patient.

TABLE 4
Average Spot-Forming Unit Values

| | C1D1 | C1D8 | C2D8 | C4D8 |
|---|---|---|---|---|
| Vehicle | 0.0 | 0.0 | 0.0 | 0.0 |
| KLH | 7.8 | 2.2 | 1.1 | 3.3 |
| Peptides 1-5 | 0.0 | 1.1 | 5.6 | 60.0 |
| Peptides 6-10 | 0.0 | 4.4 | 30.0 | 66.7 |
| Peptides 11-15 | 0.0 | 10.0 | 26.7 | 156.7 |
| Peptides 16-20 | 0.0 | 3.3 | 1.1 | 3.3 |
| Peptides 21-25 | 4.4 | 10.0 | 0.0 | 1.7 |

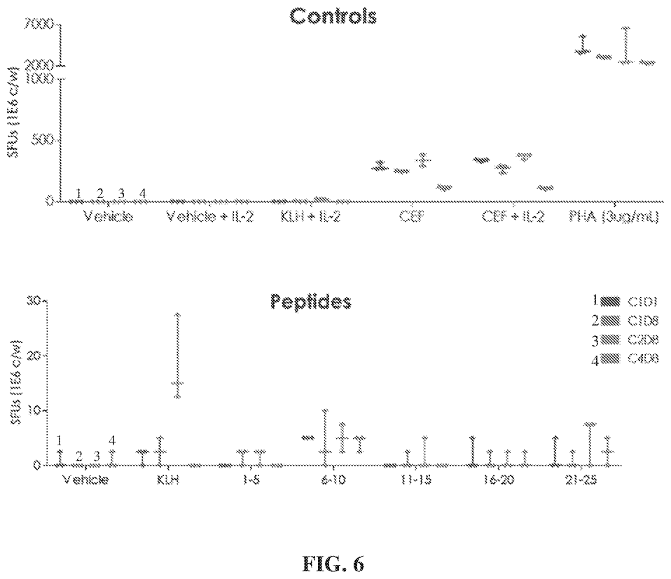

As shown in FIG. 6, for a patient with NSCLC at the 0.39 mg dose level, antigen-specific T cell responses above assay LLOQ (13.5 SFU/1e10⁶) were not detected. Table 5 summarizes the average spot forming unit (SFU) values for each peptide pool at each collection point for this patient.

TABLE 5
Average Spot-Forming Unit Values

| | C1D1 | C1D8 | C2D8 | C4D8 |
|---|---|---|---|---|
| Vehicle | 0.8 | 0.0 | 0.0 | 0.8 |
| KLH | 1.7 | 2.5 | 18.3 | 0.0 |
| Peptides 1-5 | 0.0 | 1.7 | 1.7 | 0.0 |
| Peptides 6-10 | 5.0 | 4.2 | 5.0 | 4.2 |
| Peptides 11-15 | 0.0 | 0.8 | 1.7 | 0.0 |
| Peptides 16-20 | 1.7 | 0.8 | 0.8 | 0.8 |
| Peptides 21-25 | 1.7 | 0.8 | 5.0 | 2.5 |

If the two patients who responded, the 0.13 dose patient (FIG. 1) and the 0.39 melanoma patient (FIG. 5), the difference in SFU cells/well observed was over three-fold (156.7 SFU/1e10⁶ cells/well versus 35 SFU/1e10⁶). In addition, the responses detected in the 0.39 mg dose patient were observed at two time points, whereas the response was only detected after the fourth dose in the 0.13 mg dose patient. The differences and absolute magnitude and time of occurrence between the two patients' antigen-specific T cell response may be due to the higher vaccine dose used (0.39 mg versus 0.13 mg), and may indicate a correlation between dose level and magnitude of peripheral antigen-specific T cell responses. The data also indicate that a minimum of 3 out of 20 neoantigens encoded for in the vaccine were immunogenic, with a high potential of 15 out of 20. Both patients have the same tumor type (melanoma), and similarities were observed in gene expression levels of key genes between the patients' pre-treatment biopsies, as measured by RNAseq. Both patients have a relatively low GEP score in their pre-treatment tumor biopsies, as well as lower levels of PD-1 as compared to test samples and other patient samples analyzed to date.

Data from the one patient at the 1.0 mg dose has been obtained. Antigen-specific T cell responses were detected in peptide-pulsed dendritic cell: T-cell co-culture assay that were 2 to 3-fold over baseline after the fourth dose of mRNA. The subject has NSCLC.

### Clinical Data—the Combination Arm of Phase 1 Trial

Twenty patients have been treated with combination therapy (mRNA vaccine and pembrolizumab) at the following dosages of mRNA vaccine: 0.04 mg (4 patients), 0.13 mg (5 patients), 0.39 mg (7 patients), and 1.0 mg (4 patients). Of the 20 patients, eight have progressed without responding and are off study (two patients with melanoma, three patients with non-small cell lung cancer (NSCLC), two patients with bladder cancer, and one patient with microsatellite instability (MSI)-high colorectal cancer). One patient has possible progressive disease. The patient has stable target and non-target lesions and one new lesion. Three patients have stable disease; one with MSI-high prostate cancer and two with NSCLC. Two patients have had a partial response; one with small cell lung cancer (SCLC; prior chemotherapy) experienced a partial response following two cycles of combination treatment, but has subsequently progressed and discontinued treatment, while the other subject, who has bladder cancer (multiple prior therapies, disease progression without response during prior atezolizumab treatment) had a partial response following two cycles of combination treatment and is continuing in the study. One subject, with MSI-high colorectal cancer (prior chemotherapy) experienced a complete response while on pembrolizumab monotherapy before the start of the mRNA vaccine dosing regimen. The subject remains on combination therapy. One patient, having SCLC (prior chemotherapy), was not evaluable after the first on-treatment radiological assessment because one of three target lesions was not able to be evaluated. The other two target lesions have decreased by 41% and 24%, and one non-target lesion in the brain is no longer present. The other non-target lesions, located in the bone, are still present. The four remaining patients have not yet had their first on-treatment radiological assessments. The patients have been dosed up to 0.39 mg of mRNA vaccine and fixed 200 mg doses of pembrolizumab. There have been no dose-limiting toxicities or significant related toxicities observed in the patients.

The pembrolizumab was administered while the mRNA cancer vaccine is being manufactured for each subject. The first dose of pembrolizumab is termed P1D1 and may be administered as soon as the subject is enrolled. Once the subject's mRNA cancer vaccine is manufactured and available, the pembrolizumab Combination Therapy Phase commenced. The first dose of mRNA cancer vaccine (C1D1) was administered with the next dose of pembrolizumab in order to achieve synchronous combination dosing in 21 day cycles. C1D1 represents the first dose of the cancer vaccine (C1) given on Day 1 (D1) of the 21-day cycle.

Typically the first dose of mRNA cancer vaccine (C1D1) will be administered with the third dose of pembrolizumab (P3D1, the third dose of pembrolizumab administered on Day 1 of the 21-day cycle). The third pembrolizumab dose (P3D1) may be moved back if required, by a maximum of 14 days (to occur up to 35 days after the second pembrolizumab dose) in order to accommodate the sequencing and manufacture of mRNA cancer vaccine prior to P3D1. This is only in the event that mRNA cancer vaccine is not available on or before P3D1. In the event of any NGS or vaccine manufacture issues then the first dose of mRNA cancer vaccine may also be delayed until the fourth dose of pembrolizumab (P4D1). In the event that mRNA cancer vaccine is not available by P4D1 the specific situation must be discussed with the Sponsor (later start to mRNA cancer vaccine dosing may be permissible on a case by case basis), and in the unlikely event that mRNA cancer vaccine cannot be manufactured for an individual subject, the subject may continue on pembrolizumab monotherapy.

In the event that the time to manufacture mRNA cancer vaccine is shortened, an earlier first dose of mRNA cancer vaccine is allowable (e.g., concurrent with the second dose of pembrolizumab [P2D1]). Subjects will receive a maximum of 9 cycles of mRNA cancer vaccine and pembrolizumab combination therapy. Thereafter, subjects may continue on pembrolizumab (during the pembrolizumab Monotherapy Continuation Phase) subjects will receive their assigned dose on Day 1 of each 21-day cycle, on days that are aligned with the FDA-approved dosing schedule used for pembrolizumab. The only exception to this 21-day dosing cycle is that the third pembrolizumab dose on P3D1 may be moved back if required, by a maximum of 14 days (to occur up to 35 days after the second pembrolizumab dose) in order to accommodate the sequencing and manufacture of mRNA cancer vaccine prior to P3D1. This is only in the event that mRNA cancer vaccine is not available on or before P3D1. A maximum of nine cycles of mRNA cancer vaccine are planned in order to boost the intended tumor-killing T cell population over an extended period of time by promoting optimal development of T effector memory cells.

In the combination therapy, 3 of 3 patients who received 0.13 mg one preparation of vaccines had detectable immunogenicity, one greater than LLOQ; and two greater than LOD. 1 of 3 patients who received 0.13 mg or another preparation of vaccines had detectable immunogenicity.

### Expansion Phases of Clinical Study

In a further study, the safety, tolerability, and efficacy of the mRNA vaccine in combination with pembrolizumab in programmed cell death protein 1 (PD1)/programmed death ligand 1 (PD-L1) inhibitor-naïve patients with either head and neck squamous cell carcinoma, bladder cancer, or microsatellite stable colorectal cancer, are examined. In an additional study, the mRNA vaccine is assessed in combination with pembrolizumab in a small cohort of 6 to 10 patients with adjuvant melanoma.

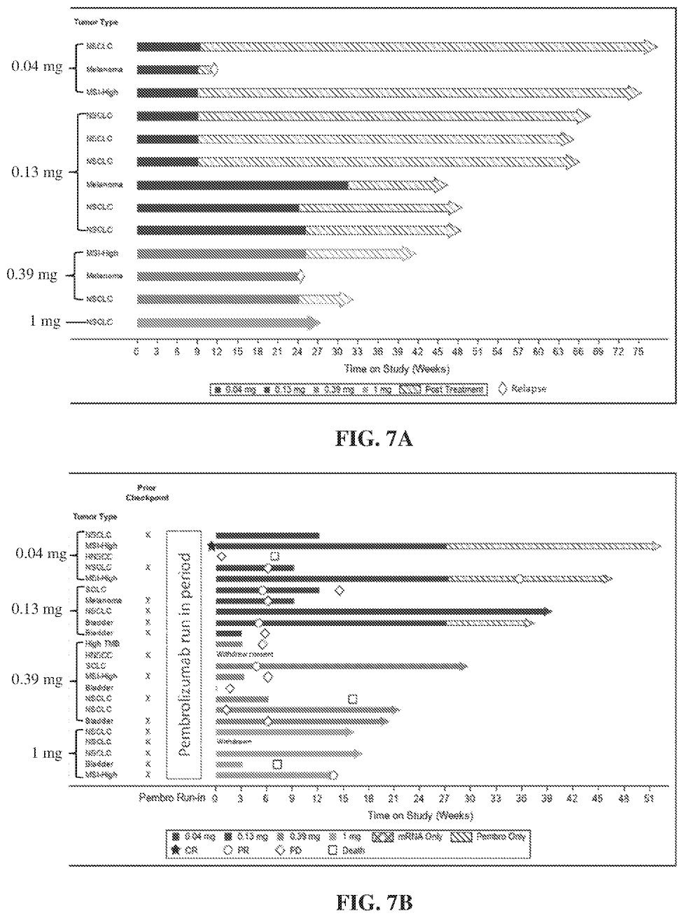

The Phase 1, Open-Label, Multicenter Study to Assess the Safety, Tolerability, and Immunogenicity of mRNA Vaccine alone in subjects with resected solid tumors and in combination with pembrolizumab in subjects with unresectable solid tumors was conducted. As seen in FIG. 7A, 13 adjuvant patients have been treated with mRNA. All 13 patients have completed full course of vaccination per protocol. 11 patients remain disease free up to 75 weeks on study. As seen in FIG. 7B, 20 out of 23 advanced/metastatic patients have been treated with mRNA/pembrolizumab combination. 1 patient with MSI-High CRC had a CR on pembrolizumab monotherapy prior to vaccination and remains a CR out to 51 weeks on study. 5 patients had PR including two patients who both had progressed on previous checkpoint inhibitor and a patient who had to discontinue pembrolizumab due to irAE and is receiving mRNA monotherapy. A patient was deemed to have experienced pseudoprogression and remains on study treatment. One patient had a new lesion, which improved at subsequent evaluation and patient remains on study treatment. Seven patients had stable disease, and 10 patients remain on study treatment with ongoing clinical benefit.

TABLE 6

| Responses in patients receiving combination | Total (N = 20) |
|---|---|
| Best Overall Response | |
| Complete Response (CR) | 1 |
| Partial Response (PR) | 5 |
| Stable Disease (SD) | 6 |
| Progressive Disease (PD) | 8 |

A patient with bladder urothelial carcinoma was previously treated with radical cystoprostatectomy, cisplatin/gemcitabine, HRS7-SN38, four cycles of atezolizumab, and vinorelbine along with a frontal lobe resection with radiation for a brain metastasis. After 2 cycles of monotherapy pembrolizumab and 2 cycles of mRNA/pembrolizumab combination, the patient had a partial response and has continued to improve while on study. A patient with small cell lung carcinoma was previously treated with chemoradiation and prophylactic cranial irradiation. After 2 cycles of monotherapy pembrolizumab and 2 cycles of mRNA/pembrolizumab combination, the patient had a partial response. The patient had progressive disease at subsequent evaluation. A patient with small cell lung carcinoma was previously treated with cisplatin/etoposide and doxorubicin/lurbinectedin. After 1 cycle of pembrolizumab run-in, patient experienced an irAE which led to treatment with monotherapy mRNA. The patient had a partial response at the first post-baseline scan and remains on the study with a PR.

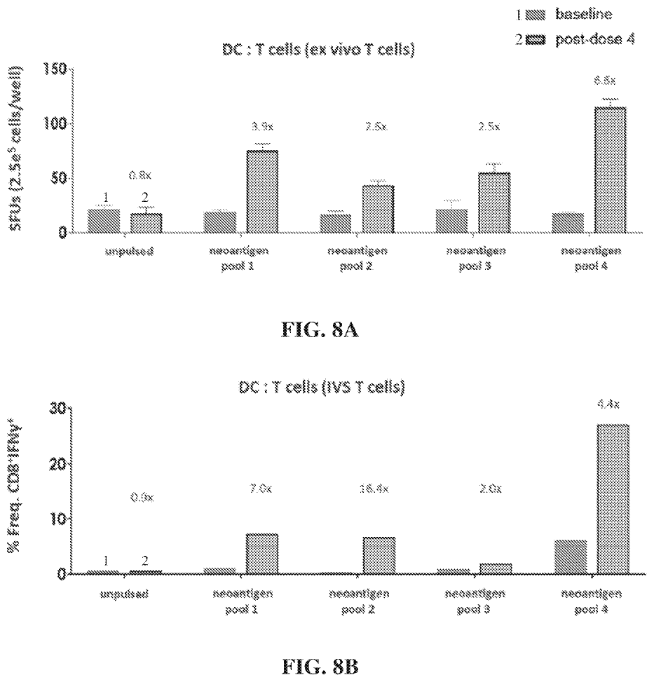

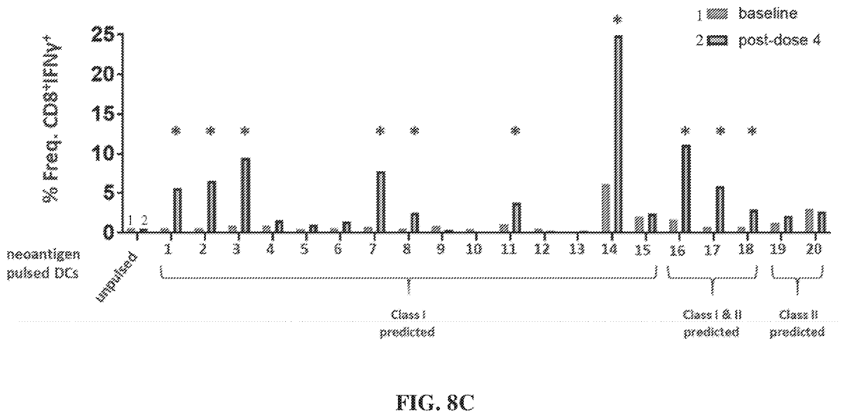

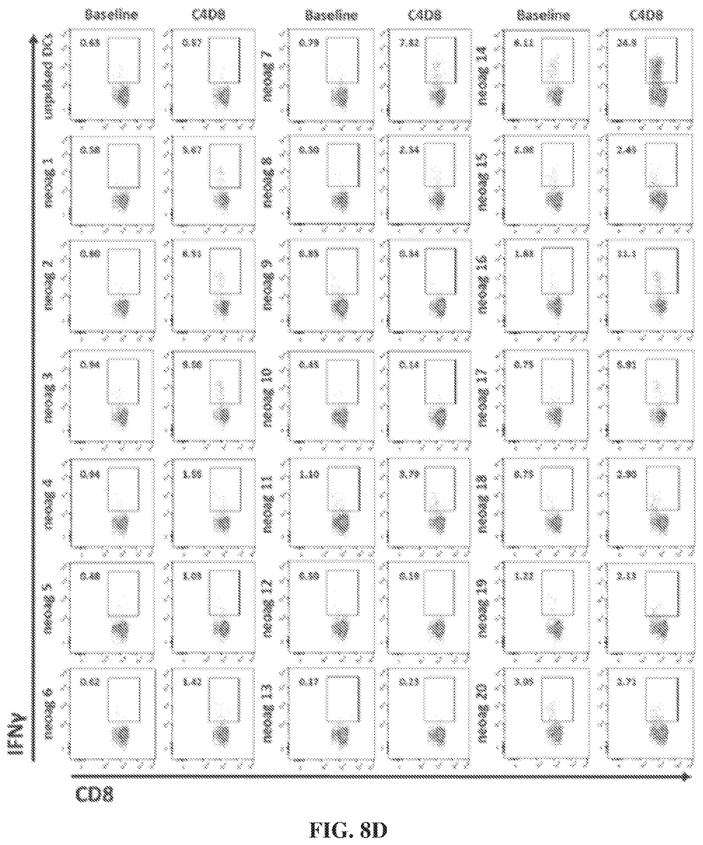

One patient, who had NSCLC, was dosed with 1 mg of vaccine monotherapy. This patient underwent apheresis at baseline and 7d post 4th dose, has received all nine doses of vaccine, and has not relapsed. Neoantigen specific T-cell responses were evaluated in this patient by two methods that both used autologous monocyte derived dendritic cells (DCs) to present neoantigens to T-cells (FIGS. 8A-8D). In the first, pulsed DC restimulation ELISpot, PBMCs were cultured for six days to yield two separate populations: mature DCs and T-cells. The DCs are exposed to an antigen pulse and then co-cultured with the T-cells. In this way, the neoantigens are presented more naturally through autologous DCs to T-cells. This is an ex vivo measurement of antigen-specific responses, as the T-cells are not expanded. Responses may be monitored by IFNγ ELISpot or flow cytometry. In contrast, the second method, pulsed DC restimulation ELISpot with in vitro stimulation (IVS) of T-cells, includes a T-cell expansion step, allowing for a sensitive measure of neoantigen-specific responses. As shown in FIG. 8A, increases in ex vivo (unexpanded) T-cell responses were detected against all neoantigen pulsed DC pools post-vaccination. Likewise, increases in in vitro stimulated (IVS, expanded) T-cell responses were also detected against all neoantigen pulsed DC pools post-vaccination (FIG. 8B). Flow cytometry was also performed to examine the in vitro stimulated (IVS, expanded) T-cell responses. The flow cytometry plots (FIG. 8D) show increases in the frequency percentage of CD8 cells producing IFNγ seven days after the fourth vaccine dose to multiple antigens. FIG. 8C shows increases in neoantigen-specific CD8 T-cells detected post-vaccination against 10 out of 18 class I targeted neoantigens. The asterisks indicate increases of three or more times the baseline percentage. All positive CD8 T-cell responses post-vaccination were to neoantigens with high predicted binding affinities of less than 500 nm.

mRNA is well tolerated at all dose levels studied with no DLTs reported. No mRNA related grade ¾ AE or SAE was reported. Clinical responses have been seen in 6 out of 20 patients treated with mRNA-/pembrolizumab combination. Of these 6 patients, 2 responses have been seen in a patient previously treated with PD-(L)1 inhibitor. Neoantigen specific CD8 T-cell responses were detected to 10 out of 18 class I neoantigens included in a patient vaccine, the first patient dosed at 1 mg who underwent apheresis. 100% of positive CD8 T-cell responses post vaccination were to neoantigens with a high predicted binding affinity of <500 nm.

### Phase 2 Clinical Study

Approximately 150 eligible subjects are randomly assigned in a 2:1 ratio to a combination arm of the study (approximately 100 subjects receive the mRNA vaccine and pembrolizumab) or to the control arm of the study (approximately 50 subjects receive pembrolizumab alone). Subjects with completely resected cutaneous melanoma (Stage IIIB, if relapsed within 3 months following initial surgery of curative intent and underwent a second surgery of curative intent prior to entering the study; Stage IIIC, Stage IIID, and Stage IV) are enrolled. Prior to enrollment, subjects must have had complete resection (surgery of curative intent) within 13 weeks prior to enrollment and must be disease-free at study entry.

The subjects in the combination arm undergo a pembrolizumab run in period, typically 2 pembrolizumab cycles, while the mRNA vaccine is being manufactured. Once the subject's mRNA vaccine is available, the combination treatment period commences. The first dose of the mRNA vaccine is administered with the next dose of pembrolizumab in order to achieve synchronous combination dosing in 21 day cycles. Pembrolizumab is administered as a 200 mg dose via a 30-minute intravenous infusion once every 21 days for up to 18 cycles (approximately one year), while the mRNA vaccines are administered as 1 mg doses via intramuscular injection once every 21 days for 9 doses.

For example, the first dose of the mRNA vaccine is administered with the third dose of pembrolizumab (the third pembrolizumab dose may be moved later, by a maximum of 14 days—i.e., up to 35 days after the second pembrolizumab dose—if necessary to accommodate the manufacture of the mRNA vaccine). The first dose of the mRNA vaccine may also be delayed until the fourth or fifth dose of pembrolizumab (for example, due to sequencing or manufacturing delay). In the unlikely event that the mRNA vaccine cannot be provided by the time of the fifth dose of pembrolizumab, the patient does not receive the mRNA vaccine but may continue on pembrolizumab monotherapy. In total, patients receive nine doses of the mRNA vaccine.

All patients, regardless of study arm, may continue on pembrolizumab until disease recurrence, unacceptable toxicity, or 18 total cycles (approximately 1 year of treatment), whichever occurs sooner.

The primary endpoint of the trial is recurrence-free survival (RFS), defined as the time between the first dose of pembrolizumab and the date of first recurrence (local, regional, or distant metastasis), new primary melanoma, or death (from any cause), whichever occurs first. Patients will be assessed by radiological imaging (computed tomography or magnetic resonance imaging). Imaging will occur during the screening phase (a baseline scan to confirm eligibility and disease-free status), during the treatment phase (every 12 weeks (±7 days) from the first dose of pembrolizumab for 12 months, including patients who discontinue pembrolizumab before the full 12 months), and the follow-up phase (every 12 weeks (±14 days) from 12 to 24 months (from the first dose of pembrolizumab) and every 6 months (±4 weeks) from 24 months to 3 years (from the first dose of pembrolizumab)). The patients will continue imaging until recurrence, death, start of a new anticancer therapy, withdrawal of consent to follow-up or lost to follow-up, or three years after the patient's first dose of pembrolizumab, whichever occurs first.

### Summary of Methods

The overall system to enable this unique process involves four major components that are highly integrated to ensure a robust chain of custody of subject-specific information that is used to design, manufacture and ultimately administer mRNA cancer vaccine to each individual subject. The four components include: (1) the individual subject, (2) Next Generation Sequencing (NGS), (3) the Bioinformatics System and (4) mRNA cancer vaccine production. The integration of the overall process is achieved using automated tracking software to provide connections between interfaces. This software tracks subject samples, NGS and bioinformatics data, and mRNA cancer vaccine production using a unique patient identification number (PID). For each subject, tumor and peripheral blood samples are collected and the subject is assigned a unique PID (Subject Number). The Subject Number is the primary identifier used to track each step in the process. Additional identification numbers are used to enable tracking within third party vendor systems.

Subject tumor and blood samples are sent for NGS analysis. Whole exome sequencing (WES) data is generated from both tumor and blood samples, with the blood sample serving as the germline (un-mutated) reference. Whole exome sequencing results from the blood sample are also used to determine the subject's HLA-type using an NGS-based approach adhering to the guidelines of the American Association for Histocompatibility and Immunogenetics (ASHI). The transcriptome is determined by mRNA sequencing (RNA-Seq) from the tumor sample only.

The HLA typing, WES and RNA-Seq results for each subject are provided as inputs to the automated mRNA cancer vaccine Bioinformatics System which uses the subject-specific data to determine the amino acid sequences of the highest scoring neoantigens and incorporate the top candidates into a concatemeric mRNA cancer vaccine sequence. The mRNA cancer vaccine sequence is then converted to multiple DNA nucleotide sequences that are optimized for ease of manufacturing. The optimized sequences are transferred electronically to production for manufacture of each subject-specific mRNA cancer vaccine. Upon completion of manufacturing and testing, mRNA cancer vaccine is shipped to the clinical site for administration to the specific subject. This process will take approximately 6-8 weeks from tissue acquisition to first dose.

Randomization is performed centrally at the time of enrollment via the central interactive web response system (IWRS). Patients will be stratified by the American Join Committee on Cancer stage of disease within 14 days after consent.

iRECIST Assessment of Disease: iRECIST is adapted to account for the unique tumor response seen with immunotherapeutic drugs. iRECIST is used to assess tumor response and progression, and make treatment decisions.

PBMCs from blood samples: T cell immunogenicity is used to examine immune response to mRNA cancer vaccine as a measure of activity. T cell immunogenicity will be assessed via antigen-specific T cell assays in PBMCs from blood samples in all subjects. Whole blood samples (100 mL) for T cell assays are collected from all subjects. When sampling time points coincide with dosing days, blood will be collected pre-dose for immunogenicity assessments. On average 70 million viable PBMCs can be recovered from 100 million cryopreserved PBMCs, which are typically isolated from 100 mL of whole blood. In order to best assess immunogenicity of mRNA cancer vaccine, T cell responses to each of the multiple epitopes encoded in each subject's mRNA cancer vaccine will be measured individually if possible (depending on the actual number of epitopes, and the actual volume of blood available). Approximately 70 million viable PBMCs per collection are required to perform T cell assays (which may include but are not limited to assays such as Enzyme-linked ImmunoSpot assay and flow cytometry). If viable PBMC recovery is lower than required for individual epitope analysis, alternative assay strategies may be implemented to maximally assess subject's immune response to mRNA cancer vaccine. T cell immunogenicity will be used to examine immune response to mRNA cancer vaccine as a measure of activity. T cell immunogenicity will be assessed via antigen-specific T cell assays in all parts of the study.

The following exploratory efficacy endpoints of this study may be assessed (by the Investigator per RECIST 1.1 and iRECIST) as follows: Relapse-free survival (RFS), duration of response, objective response rate, and progression-free survival.

### REFERENCES

Antonia S J, et al. J Clin Oncol. 2014; 32(5s):125736-44.
Cappuccini F, et al. Cancer Immunol Immunother. 2016; 65(6):701-13.
Carreno B M, et al. Science. 2015; 348(6236):803-8.
Castle J C, al. Cancer Res. 2012; 72(5):1081-91.
Coelho T, et al. N Engl J Med. 2013; 369(9):819-29.
Dyck, L, et al. Cancer Immunol Immunother. 2016; 65(12):1491-8.
Fitzgerald, K, et al. Lancet. 2014; 383(9911):60-8.
Langer C, et al. European Society for Medical Oncology (EMSO) Congress 2016; KEYNOTE-021.
Matsushita H, et al. Nature. 2012; 482(7385):400-4.
McGranahan N, et al. Science. 2016; 351(6280):1463-9.
Rizvi N A, et al. Science. 2015; 348(6230):124-8.
Schumacher T N and Schreiber R D. Science. 2015; 348(6230):69 74.
Sharma P and Allison J P. Science. 2015; 348(6230):56-61.
Snyder A, et al. N Engl J Med. 2014; 371(23):2189-99.
Szebeni J. Mol Immunol. 2014; 61(2):163-73.
Topalian S L, et al. Nat Rev Cancer. 2016; 16(5):275-87.
Van Allen et al. Science. 2015:350(6257):207-211.
Yadav M S, et al. Nature. 2014; 515(7528):572-6.

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety.

What is claimed is:

1. A method of treating a cancer in a human subject comprising:
administering to the human subject multiple doses of a mRNA cancer vaccine composition at a dosage of 0.04-0.13 mg, 0.13-0.39 mg, or 0.39-1.0 mg, 1.0-5.0 mg wherein the mRNA cancer vaccine composition comprises one or more mRNAs each having one or more open reading frames encoding 3-50 peptide epitopes, wherein each of the peptide epitopes are portions of personalized cancer antigens or portions of cancer hotspot antigens, formulated in a lipid nanoparticle formulation, thereby treating the cancer in the human subject.

2. The method of claim 1, wherein at least 3 doses of the cancer vaccine composition are administered to the subject.

3. The method of claim 1, wherein at least 5 doses of the cancer vaccine composition are administered to the subject.

4. The method of claim 1, wherein at least 9 doses of the cancer vaccine composition are administered to the subject.

5. The method of any one of claims 1-4, wherein the multiple doses of the cancer vaccine composition are administered 20-22 days apart.

6. The method of any one of claims 1-4, wherein the multiple doses of the cancer vaccine composition are administered 21 days apart.

7. The method of any one of claims 1-6, wherein the cancer vaccine composition comprises one mRNA having one open reading frame encoding 30-35 peptide epitopes.

8. The method of any one of claims 1-6, wherein the cancer vaccine composition comprises one mRNA having one open reading frame encoding 34 peptide epitopes.

9. The method of any one of claims 1-6, wherein the cancer vaccine composition comprises a first and second mRNA, wherein the first mRNA has one open reading frame encoding 10-20 peptide epitopes which are portions of personalized cancer antigens and wherein the second mRNA has one open reading frame encoding peptide epitopes that are portions of cancer hotspot antigens.

10. The method of claim 9, wherein the cancer hotspot antigens comprise a KRAS G12 mutation or a KRAS G13 mutation or both mutations.

11. The method of any one of claims 1-10, wherein the cancer vaccine composition is administered at a dosage of 0.04 mg to 2 mg.

12. The method of any one of claims 1-10, wherein the cancer vaccine composition is administered at a dosage of 0.04 mg to 1 mg.

13. The method of any one of claims 1-10, wherein the cancer vaccine composition is administered at a dosage of 0.39 mg to 2 mg.

14. The method of any one of claims 1-10, wherein the cancer vaccine composition is administered at a dosage of 0.39 mg to 1 mg.

15. The method of any one of claims 1-14, wherein the minimum length of any peptide epitope is 8 amino acids.

16. The method of any one of claims 1-15, wherein the maximum length of any peptide epitope is 31 amino acids.

17. The method of claim 1, wherein the one or more mRNA each comprise a 5′ UTR and/or a 3′ UTR.

18. The method of any one of claims 1-17, wherein the one or more mRNA comprise at least one chemical modification.

19. The method of claim 18, wherein the chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

20. The method of claim 18 or claim 19, wherein the one or more mRNA is fully modified.

21. The method of any one of claims 1-6 and 8-20, wherein the peptide epitopes are in the form of a concatemeric cancer antigen comprised of 30-40 peptide epitopes.

22. The method of claim 21, wherein the peptide epitopes are interspersed by cleavage sensitive sites.

23. The method of claim 21, wherein each peptide epitope is linked directly to one another without a linker.

24. The method of claim 21, wherein each peptide epitope is linked to one another with a single amino acid linker.

25. The method of claim 21, wherein each peptide epitope is linked to one another with a short peptide linker.

26. The method of claim 21, wherein each peptide epitope includes one or more SNP mutations; and/or a mutation causing a unique expressed peptide sequence.

27. The method of claim 21, wherein the mRNA encoding the peptide epitopes is arranged such that the peptide epitopes are ordered to minimize pseudo-epitopes.

28. The method of claim 21, wherein the ratio of class I MHC molecule peptide epitopes to class II MHC molecule peptide epitopes is at least 1:1, 2:1, 3:1, 4:1, or 5:1.

29. The method of any one of claims 1-28, wherein the lipid nanoparticle comprises an ionizable amino lipid.

30. The method of claim 29, wherein the lipid nanoparticle formulation comprises a molar ratio of about 20-60% ionizable amino lipid, about 5-25% non-cationic lipid, about 25-55% sterol, and about 0.5-15% PEG-modified lipid.

31. The method of claim 29, wherein the lipid nanoparticle formulation comprises an ionizable amino lipid of Compound 1, wherein the non-cationic lipid is DSPC, the structural lipid is cholesterol, and the PEG lipid is PEG-DMG.

32. The method of any one of claims 1-31, wherein the cancer vaccine composition prevents the human subject's cancer from recurring by provoking the immune system to develop a memory immune response against a cancerous tissue derived from an original lesion of the cancer.

33. The method of any one of claims 1-32, wherein the vaccine composition is administered via intramuscular injection.

34. The method of claim 33, wherein the vaccine composition is administered in 2 or more injections.

35. The method of claim 33, wherein the vaccine composition is administered in a single injection.

36. The method of any one of claims 1-35, wherein the human subject has a favorable response to the treatment method based on RECIST (Response Evaluation Criteria In Solid Tumors).

37. The method of any one of claims 1-35, wherein the human subject has a favorable response to the treatment method based on irRECIST (Immune-related Response Evaluation Criteria In Solid Tumors).

38. The method of any one of claims 1-35, wherein the personalized cancer antigens are selected based on a next generation sequencing (NGS) analysis of the human subjects DNA from a tumor sample, relative to DNA from a blood sample.

39. A method of treating a cancer in a human subject comprising:
administering to the human subject a combination therapy comprising 1) multiple doses of a mRNA cancer vaccine composition at a dosage of 0.04-0.13 mg, 0.13-0.39 mg, or 0.39-1.0 mg, 1.0-5.0 mg, wherein the cancer vaccine composition comprises one or more mRNAs each having one or more open reading frames encoding 3-50 peptide epitopes, wherein each of the peptide epitopes are portions of personalized cancer antigens or portions of cancer hotspot antigens, formulated in a lipid nanoparticle formulation, and 2) multiple doses of an anti-cancer immunotherapy thereby treating the cancer in the human subject with the combination therapy.

40. The method of claim 39, wherein the human subject is administered at least 0.04 mg of the vaccine composition.

41. The method of claim 39, wherein the human subject is administered at least 0.13 mg of the vaccine composition.

42. The method of claim 39, wherein the human subject is administered at least 0.39 mg of the vaccine composition.

43. The method of claim 39, wherein the human subject is administered at least 1.0 mg of the vaccine composition.

44. The method of any one of claims 39-43, wherein the human subject is administered less than 2.0 mg of the vaccine composition.

45. The method of any one of claims 39-44, wherein the lipid nanoparticle formulation comprises an ionizable amino lipid nanoparticle.

46. The method of claim 45, wherein the lipid nanoparticle formulation comprises a molar ratio of about 20-60% ionizable amino lipid, about 5-25% non-cationic lipid, about 25-55% sterol, and about 0.5-15% PEG-modified lipid.

47. The method of claim 46, wherein the lipid nanoparticle formulation comprises an ionizable amino lipid of Compound 1, wherein the non-cationic lipid is DSPC, the structural lipid is cholesterol, and the PEG lipid is PEG2000-DMG.

48. The method of any one of claims 39-47, wherein the anti-cancer immunotherapy is a checkpoint inhibitor.

49. The method of claim 48, wherein the checkpoint inhibitor is pembrolizumab.

50. The method of any one of claims 39-49, wherein the cancer vaccine composition is administered to the subject twice, thrice, four times, five times, six times, seven times, eight times, nine times, or 10 times.

51. The method of any one of claims 39-50, wherein the cancer vaccine composition is administered via intramuscular injection.

52. The method of any one of claims 39-51, wherein the anti-cancer immunotherapy is administered to the human subject at least 2 times.

53. The method of any one of claims 39-51, wherein the anti-cancer immunotherapy is administered to the human subject at least 3 times.

54. The method of any one of claims 39-51, wherein the anti-cancer immunotherapy is administered to the human subject at least 9 times.

55. The method of claim 52, wherein the anti-cancer immunotherapy is administered to the human subject at least 2 times prior to the administration of the cancer vaccine composition.

56. The method of claim 53, wherein the third, fourth or fifth dose of anti-cancer immunotherapy is administered to the human subject on the same day as a first dose of the cancer vaccine composition.

57. The method of claim 56, wherein subsequent doses of the anti-cancer immunotherapy and the cancer vaccine composition are administered to the human subject on the same day.

58. The method of claim 57, wherein the anti-cancer immunotherapy is administered after the cancer vaccine composition.

59. The method of any one of claims 39-58, wherein the cancer is selected from the group consisting of small cell lung cancer, urothelial cancer, colorectal cancer, endometrial cancer, stomach cancer, gastro-esophageal junction cancer melanoma, bladder carcinoma, HPV negative HNSCC, NSCLC, SCLC, MSI (microsatellite)-High tumors, or TMB (tumor mutational burden)-High cancers.

60. The method of any one of claims 39-58, wherein the anti-cancer immunotherapy is administered every 20-24 days.

61. The method of any one of claims 39-58, wherein the anti-cancer immunotherapy is administered every 21 days.

62. The method of any one of claims 39-61, wherein the anti-cancer immunotherapy is administered at a dose of 150-250 mgs.

63. The method of any one of claims 39-61, wherein the anti-cancer immunotherapy is administered at a dose of 200 mgs.

64. The method of any one of claims 39-63, wherein the cancer is a PD-L1 negative tumor.

65. The method of any one of claims 39-55, wherein the anti-cancer immunotherapy is administered within 7 days of the mRNA cancer vaccine.

66. The method of any one of claims 39-55, wherein the anti-cancer immunotherapy is administered within 2-3 weeks of the mRNA cancer vaccine.

67. The method of any one of claims 39-66, wherein the cancer has a high tumor mutational burden.

68. The method of any one of claims 39-66, wherein the subject is selected for treatment based on a threshold tumor mutational burden.

69. The method of any one of claims 39-66, wherein the subject is selected for treatment based on a threshold microsatellite instability (MSI) value.

70. The method of any one of claims 39-66, wherein the subject is selected for treatment based on a threshold T-cell inflamed gene expression profile (GEP).

71. The method of claim 70, wherein the GEP comprises PD-1, PD-L1 and PD-L2.

72. The method of claim 71, wherein the subject is selected for treatment based on high levels of PD-L1 and PD-L2.

73. The method of claim 71, wherein the subject is selected for treatment based high levels of PD-L1 and PD-1.

74. The method of any one of claims 39-66, wherein the subject is selected for treatment based on a threshold GEP and TMB.

75. The method of claim 48, wherein the checkpoint inhibitor targets PD1, PD-L1, CTLA4, TIM-3, VISTA, A2AR, B7-H3, B7-H4, BTLA, IDO, KIR, LAG3, or a combination thereof.

76. The method of claim 48, wherein the checkpoint inhibitor is an antibody.

77. The method of claim 76, wherein the checkpoint inhibitor is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof.

78. The method of claim 48, wherein the checkpoint inhibitor is an anti-PD-L1 antibody selected from atezolizumab, avelumab, or durvalumab.

79. The method of claim 48, wherein the checkpoint inhibitor is an anti-CTLA-4 antibody selected from tremelimumab or ipilimumab.

80. The method of claim 48, wherein the checkpoint inhibitor is an anti-PD1 antibody selected from nivolumab or pembrolizumab.
